(19) United States
(12) Patent Application Publication
(10) Pub. No.: US 2021/0090690 A1
(43) Pub. Date: Mar. 25, 2021

(54) MOLECULAR DESIGN USING REINFORCEMENT LEARNING

(71) Applicant: BENEVOLENTAI TECHNOLOGY LIMITED, London (GB)

(72) Inventors: Dean PLUMBLEY, London (GB); Marwin Hans Siegfried SEGLER, Southsea Hampshire (GB)

(73) Assignee: BENEVOLENTAI TECHNOLOGY LIMITED, London (GB)

(21) Appl. No.: 17/041,573

(22) PCT Filed: Mar. 29, 2019
(86) PCT No.: PCT/GB2019/050925

§ 371 Date: Sep. 25, 2020

(30) Foreign Application Priority Data
Mar. 29, 2018 (GB) 1805300.9

(51) Int. Cl. G16C 20/50; G16C 20/70

(52) U.S. Cl. CPC G16C 20/50; G16C 20/70

(57) ABSTRACT

Method(s), apparatus and system(s) are provided for designing a compound exhibiting one or more desired property(ies) using a machine learning (ML) technique. This may be achieved by generating a second compound using the ML technique to modify a first compound based on the desired property(ies) and a set of rules for modifying compounds; scoring the second compound based on the desired property(ies); determining whether to repeat the generating step based on the scoring; and updating the ML technique based on the scoring prior to repeating the generating step.

The present application relates to a apparatus, systems and method(s) for designing a compound exhibiting one or more desired property(ies) using a machine learning (ML) techniques.

### BACKGROUND

Informatics is the application of computer and informational techniques and resources for interpreting data in one or more academic and/or scientific fields. Cheminformatics' and bioinformatics includes the application of computer and informational techniques and resources for interpreting chemical and/or biological data. This may include solving and/or modelling processes and/or problems in the field(s) of chemistry and/or biology. For example, these computing and information techniques and resources may transform data into information, and subsequently information into knowledge for rapidly creating compounds and/or making improved decisions in, by way of example only but not limited to, the field of drug identification, discovery and optimization.

Machine learning techniques are computational methods that can be used to devise complex analytical models and algorithms that lend themselves to solving complex problems such as creation and prediction of compounds with desired characteristics and/or property(ies). There are a myriad of ML techniques that may be used or selected for generating compounds, but none which could be used to generate or optimise a compound based on a set of desired characteristics or properties. There is a desire to use ML techniques to allow researchers, data scientists, engineers, and analysts to make rapid improvements in the field of drug identification, discovery and optimisation.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides method(s), apparatus and system(s) for designing and generating candidate molecules/compounds exhibiting desired properties using machine learning (ML) techniques. The ML technique(s) may start from an initial compound and learn how to perturb the compound until a compound with desired properties is achieved or a stopping criterion is achieved. The approach described herein does not require manual intervention and may be performed automatically. The sequence of rules, actions and/or perturbations performed that results in the final compound may also be output and may provide a researcher with additional insight and/or evidence as to how the compound may be formed and that the compound has the desired property(ies) and/or characteristics.

In a first aspect, the present disclosure provides a computer-implemented method for designing a compound exhibiting one or more desired property(ies) using a machine learning, ML, technique, the method comprising: generating a second compound using the ML technique to modify a first compound based on the desired property(ies) and a set of rules for modifying compounds; scoring the second compound based on the desired property(ies); determining whether to repeat the generating step based on the scoring; and updating the ML technique based on the scoring prior to repeating the generating step.

Preferably, determining whether to repeat the generating step is based on the scoring indicating the second compound is closer to a compound that exhibits the desired property(ies).

Preferably, determining whether to repeat the generating step is based on the scoring indicating the second compound exhibits the desired property(ies).

Preferably, determining whether to repeat the generating step further comprises determining whether a predetermined number of iterations of repeating the generating step has been achieved.

Preferably, determining whether to repeat the generating step further comprises determining, based on the second compound exhibiting at least one or more of the desired property(ies), whether any further improvements to the second compound are possible.

Preferably, generating a second compound further comprises generating a set of second compounds; and scoring the set of second compounds based on the desired property(ies).

Preferably, the method further comprising: ranking the set of second compounds based on the scoring, wherein generating a second compound further comprises generating further second compounds based on the topmost ranked set of second compounds.

Preferably, the set of rules further comprises data representative of one or more action(s) associated with modifying compounds.

Preferably, the one or more action(s) comprises one or more action(s) from the group of: an action corresponding to adding a compound fragment or one or more atoms to the compound; an action corresponding to removing a compound fragment or one or more atoms of the compound; an action corresponding to breaking or removing a bond between atoms of a compound; an action corresponding to adding or reforming a bond between atoms of a compound; any other action associated with modifying a compound to form another compound; and any other action associated with modifying a compound to form a different compound.

Preferably, the set of rule(s) and/or the one or more action(s) are selected to conform to required structural, physical and/or chemical constraints that ensure any modification(s) to the compound and/or subsequent modified compounds are feasible.

Preferably, the set of rule(s) and/or the one or more action(s) are based on a set of relevantly chemical groups comprising one or more of: one or more of atom(s); one or more molecule(s); one or more other compound(s); one or more compound fragment(s); one or more bond(s); one or more functional group(s); and one or more chemically relevant aspects of the compound and the like.

Preferably, generating a second compound further comprises generating a tree data structure comprising a plurality of nodes and a plurality of edges, wherein each edge connects a parent node to a child node, wherein a parent node represents a compound and each edge from a parent node to a child node represents an action of the plurality of actions performed on the compound of the parent node that results in the compound of the child node, wherein the root node of the tree is the first compound and subsequent nodes correspond to a set of second compound(s).

Preferably, the method further comprising expanding the tree data structure based on scoring one or more nodes corresponding to the set of second compound(s).

Preferably, the method further comprising performing a tree search on the tree data structure to generate a set of second compounds based on a set of one or more actions from the plurality of actions.

Preferably, generating one or more second compound(s) further comprises: mapping, by the ML technique, the first compound and a set of actions to an N-dimensional action space; selecting, by the ML technique, a subset of actions in the N-dimensional action space that are nearest neighbour to the first compound when mapped in the N-dimensional action space; and applying the subset of actions in the N-dimensional space to the first compound to generate a set of one or more second compound(s).

Preferably, generating the set of second compound(s) further comprises selecting nodes associated with the selected set of actions for inclusion into the tree data structure.

Preferably, the desired property(ies) includes one or more from the group of: the compound docking with another compound to form a stable complex; the particular property is associated with a ligand docking with a target protein, wherein the compound is the ligand; the compound docking or binding with one or more target proteins; the compound having a particular solubility or range of solubilities; and any other property associated with a compound that can be simulated using computer simulation(s) based on physical movements of atoms and molecules.

Preferably, the score comprises a certainty score, wherein one or more of the second compound(s) has a upper certainty score when those compounds substantially exhibit all of the one or more desired property(ies), one or more of the second compound(s) have a lower certainty score when those compound(s) substantially do not exhibit some of the one or more desired property(ies), and one or more of the second compound(s) have an uncertainty score between the upper certainty score and lower certainty score when those compounds substantially exhibit some of the one or more desired property(ies).

Preferably, the certainty score is a percentage certainty score, wherein the upper certainty score is 100%, the lower certainty score is 0%, and the uncertainty score is between the upper and lower certainty scores.

Preferably, generating the one or more second compound(s) further comprises using a reinforcement learning, RL, technique for selecting the one or more of a plurality of rules for modifying the first compound into a second compound.

Preferably, at least part of the scoring is performed using one or more ML technique(s).

Preferably, the ML technique comprises at least one ML technique or combination of ML technique(s) from the group of: a recurrent neural network configured for predicting, starting from a first compound, a second compound exhibiting a set of desired property(ies); convolutional neural network configured for predicting, starting from a first compound, a second compound exhibiting a set of desired property(ies); reinforcement learning algorithm configured for predicting, starting from a first compound, a second compound exhibiting a set of desired property(ies); and any neural network structure configured for predicting, starting from a first compound, a second compound exhibiting a set of desired property(ies).

Preferably, scoring a second compound based on the desired property(ies) further comprises: analysing the second compound against each of the desired property(ies); and calculating an aggregated score for the second compound based on the analysis.

Preferably, analysing the second compound further comprises performing a computer simulation associated with one or more of the desired property(ies) for the second compound.

Preferably, analysing the second compound further comprises using a knowledge based expert to determine whether the second compound exhibits one or more of the desired property(ies).

Preferably, one or more first compound(s) are input to the ML technique when generating a second compound using the ML technique.

Preferably, generating a second compound using the ML technique further comprises generating a set of second compounds using the ML technique based on the desired property(ies) and the set of rules.

In a second aspect, the present disclosure provides an apparatus comprising a processor, a memory unit and a communication interface, wherein the processor is connected to the memory unit and the communication interface, wherein the processor and memory are configured to implement the computer-implemented method according to the first aspect, modifications thereof and/or as described herein.

In a third aspect, the present disclosure provides a computer-readable medium comprising data or instruction code, which when executed on a processor, causes the processor to implement the computer-implemented method according to the first aspect, modifications thereof and/or as described herein.

In a fourth aspect, the present disclosure provides a machine learning, ML, model comprising data representative of updating a ML technique according to the computer-implemented method according to the first aspect, modifications thereof and/or as described herein.

In a fifth aspect, the present disclosure provides a machine learning, ML, model obtained from the computer-implemented method according to the first aspect, modifications thereof and/or as described herein.

In a sixth aspect, the present disclosure provides a tangible computer-readable medium comprising data or instruction code for designing a compound exhibiting one or more desired property(ies) using a machine learning, ML, technique, which when executed on one or more processor(s), causes at least one of the one or more processor(s) to perform at least one of the steps of the method of: generating a second compound using the ML technique to modify a first compound based on the desired property(ies) and a set of rules for modifying compounds; scoring the second compound based on the desired property(ies); determining whether to generate step based on the scoring; and updating the ML technique based on the scoring prior to repeating the generating step.

Preferably, the computer-readable medium further comprising data or instruction code, which when executed on a processor, causes the processor to implement one or more steps of the computer-implemented method according to the first aspect, modifications thereof and/or as described herein.

In a seventh aspect, the present disclosure provides a system for designing a compound exhibiting one or more desired property(ies) using a machine learning, ML, technique, the system comprising: a compound generation module configured for generating a second compound using the ML technique to modify a first compound based on the desired property(ies) and a set of rules for modifying compounds; a compound scoring module configured for scoring the second compound based on the desired property(ies); a decision module configured for determining whether to repeat the generating step based on the scoring; and an update ML module configured for updating the ML technique based on the scoring prior to repeating the generating step.

Preferably, the compound generation module, the compound scoring module, the decision module, and the update ML module are further configured to implement the computer-implemented method according to the first aspect, modifications thereof and/or as described herein.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a flow diagram illustrating an example process for designing a compound exhibiting one or more desired property(ies) according to the invention;

FIG. 1b is a schematic diagram illustrating an example apparatus for designing a compound exhibiting one or more desired property(ies) according to the invention;

FIG. 2a is a flow diagram illustrating an example process for an ML technique to generate a compound exhibiting one or more desired property(ies) according to the invention;

FIG. 2b is a schematic diagram illustrating an example apparatus for an ML technique to generate a compound exhibiting one or more desired property(ies) according to the invention;

FIG. 3a is a schematic diagram illustrating an example tree-based data structure for use by an ML technique according to the invention;

FIG. 3b is a flow diagram illustrating an example process for a tree-based ML technique to generate a compound exhibiting one or more desired property(ies) according to the invention;

FIGS. 3c-3d are a schematic diagrams illustrating the operation of an example tree-based ML technique based on the process of FIG. 3b according to the invention;

FIG. 4a is a flow diagram illustrating an example process for a encoding based ML technique to generate a compound exhibiting one or more desired property(ies) according to the invention;

FIGS. 4b-4e are a schematic diagrams illustrating the operation of the process of FIG. 4a to generate one or more candidate compound(s) exhibiting one or more desired property(ies) according to the invention;

FIG. 5a is a flow diagram illustrating an example process for an tree-encoding ML technique based on FIGS. 3a-4e to generate one or more candidate compound(s) exhibiting one or more desired property(ies) according to the invention;

FIG. 5b is a schematic diagram illustrating the operation of the example process of FIG. 5a to generate one or more candidate compounds exhibiting one or more desired property(ies) according to the invention;

FIG. 6a is a schematic diagram of a computing system and device according to the invention; and

FIG. 6b is a schematic diagram of a system according to the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The inventors have advantageously found iterative and automatic approaches using ML technique(s) for designing and generating candidate molecules/compounds exhibiting desired properties based on starting from an initial compound and perturbing it until a compound with desired properties can be achieved. This approach does not require manual intervention and may be performed automatically. The sequence of rules, actions and/or perturbations performed that results in the final compound may also be output and provide a researcher with additional insight and/or evidence as to how the compound may be formed and that the compound has the desired property(ies) and/or characteristics.

For example, reinforcement learning (RL) techniques may be applied that use one or more ML technique(s), by way of example only but are not limited to, neural networks to design and generate new molecules/compounds exhibiting one or more desired property(ies). The RL technique uses an ML technique to iteratively generate a sequence of actions for modifying an initial compound molecule/compound into another molecule/compound that may exhibit the desired properties. The ML technique may be configured to apply all known or possible actions it can take (e.g. add atom(s), break bonds, take away atoms etc.) to the initial molecule/compound or fragment(s) thereof and desired properties to output one or more possible candidate compounds.

Each candidate compound may be scored based on, by way of example only but it not limited to, atomistic computer simulations (e.g. molecular dynamics®) and/or knowledge based experts, or one or more ML techniques trained for scoring a compound against one or more desired properties, to determine whether the candidate compound is already known and how close it exhibits the desired property(ies). The RL technique updates or adapts the ML technique based on the scoring. The update of a ML technique may include, by way of example only but is not limited to, updating or adapting the parameters, coefficient(s) and/or weight(s) of the ML technique. During the update, the RL technique may penalise the ML technique if the desired properties are further away from the starting molecule/compound or if the modified molecule/compound is too big/small, and/or any other undesirable quality or difference. The RL technique may reward the ML technique if the modified molecule exhibits properties closer to the desired properties that are required. The RL technique then re-iterates the design process, which may include the ML technique starting again with the initial compound and/or starting with one of the output candidate compounds, and applying another sequence of actions to get to another modified molecule/compound. The RL technique's iterative process may complete, by way of example only but not limited to, when either a maximum number of iterations has occurred, there are no further significant improvements in candidate compounds (e.g. seen when the scoring plateaus compared with previous iterations), when the scoring indicates one or more candidate compounds exhibit the desired properties and/or there are no further significant improvements to the candidate compounds.

A compound (also referred to as one or more molecules) may comprise or represent a chemical or biological substance composed of one or more molecules (or molecular entities), which are composed of atoms from one or more chemical element(s) (or more than one chemical element) held together by chemical bonds. Example compounds as used herein may include, by way of example only but are not limited to, molecules held together by covalent bonds, ionic compounds held together by ionic bonds, intermetallic compounds held together by metallic bonds, certain complexes held together by coordinate covalent bonds, drug compounds, biological compounds, biomolecules, biochemistry compounds, one or more proteins or protein compounds, one or more amino acids, lipids or lipid compounds, carbohydrates or complex carbohydrates, nucleic acids, deoxyribonucleic acid (DNA), DNA molecules, ribonucleic acid (RNA), RNA molecules, and/or any other organisation or structure of molecules or molecular entities composed of atoms from one or more chemical element(s) and combinations thereof.

Each compound has or exhibits one or more property(ies), characteristic(s) or trait(s) or combinations thereof that may determine the usefulness of the compound for a given application. The property of a compound may comprise or represent data representative or indicative of a particular behaviour/characteristic/trait and/or property of the compound when the compound undergoes a reaction. The desired property(ies) of a compound may comprise or represent data representative of a list of one or more, or a multiple of, characteristics, traits and/or properties that a compound is desired to exhibit or be associated with. A compound may exhibit or be associated with one or more properties, which may include by way of example only but is not limited to, various aspects of one or more characteristics, traits and/or properties from the group of: an indication of the compound docking with another compound to form a stable complex; an indication associated with a ligand docking with a target protein, wherein the compound is the ligand; an indication of the compound docking or binding with one or more target proteins; an indication of the compound having a particular solubility or range of solubilities; an indication of the compound having particular electrical characteristics; an indication of the compound having a toxicity or range of toxicities; any other indication of a property or characteristic associated with a compound that can be simulated using computer simulation(s) based on physical movements of atoms and molecules; any other indication of a property or characteristic associated with a compound that can be tested by experiment or measured. Further examples of one or more compound property(ies), characteristic(s), or trait(s), may include, by way of example only but are not limited to, various aspects of one or more of: partial coefficient (e.g. Log P), distribution coefficient (e.g. Log D), solubility, toxicity, drug-target interaction, drug-drug interaction, off-target drug effects, cell penetration, tissue penetration, metabolism, bioavailability, excretion, absorption, distribution, drug-protein binding, drug-protein interaction, drug-lipid interaction, drug-DNA/RNA interaction, metabolite prediction, tissue distribution and/or any other suitable property, characteristic and/or trait in relation to a compound.

A compound exhibiting or being associated with a property may be represented by a property value or property score for that compound, the property value or property score may comprise or include data representative of or indicative of whether the compound exhibits or is associated with a particular behaviour/characteristic/trait of the property when the compound undergoes a reaction when being tested for the property. The compound may be tested in relation to the property via laboratory experimentation, computer simulation (e.g. physical or atomistic computer simulation), or via an machine learning model(s) configured for predicting whether a compound exhibits or is associated with the property being tested. Thus, the property value or property score data representative or indicative of the compound exhibiting or being associated with the property may be based on measurement values, simulation result values or data, and/or ML model(s) output values and/or prediction result values/scores and/or data and the like. A property value/score for a compound in relation to a property may comprise or represent data representative or indicative of whether the compound exhibits or is associated with the property. The property value/score may include, by way of example only but is not limited to, data representative of any one or more continuous property value(s)/score(s) (e.g. non-binary values), one or more discrete property value(s)/score(s) (e.g. binary values or integer values), one or more range(s) of continuous property values/scores, one or more range(s) of discrete property value(s)/score(s), a series of property value(s)/score(s), one or more string(s) of property values, measurement values associated with the reaction, or any other suitable data representation of a property value/score representing whether or how closely a compound exhibits or is associated with the property and the like. For example, a compound may undergo a reaction associated with a property and, based on measurements or simulations, be assigned a property value or score that is representative of whether or how closely the compound exhibits or is associated with the property. The property value/score may be based on measurement data or simulation data associated with the reaction and/or the particular property.

Thus, a compound may be scored against one or more desired property(ies) based on one or more property value(s)/score(s) that may be determined for the compound. The compounds overall property score for a desired set of property(ies) may be based on a combination of the individual property value(s)/score(s) of the desired set of property(ies) that the compound may be tested against. The compound may be tested against each individual property of the set of desired properties using, by way of example only but is not limited to, laboratory experimentation, computer simulation, ML model prediction and/or any other method for determining whether a compound exhibits one or more property(ies) and the like. For example, the combination of property value(s)/score(s) may be a weighted combination of the individual properties of the set of desired property(ies). The resulting overall compound property score that may be assigned to the compound gives an indication of whether, or how closely, that compound is associated with or exhibits the set of desired property(ies).

A rule for modifying a compound may comprise or represent data representative of any principle, operation, regulation, procedure, action or any other command, code or instruction or data format that may be used to describe modifying a compound from a first compound to a second compound. A set of rules may comprise or represent data representative of one or more rules or a plurality of rules for modifying compounds. For example, a set of rules for modifying compounds may comprise or represent data representative of one or more action(s) associated with modifying compounds such as, by way of example only but not limited to, one or more rules or action(s) from the group of: a rule or an action corresponding to adding a chemical element, a compound fragment or one or more atoms to the compound; a rule or an action corresponding to removing a chemical element, a compound fragment or one or more atoms of the compound; a rule or an action corresponding to breaking or removing a bond between atoms of a compound; a rule or an action corresponding to adding or reforming a bond between atoms of a compound; any other rule or any other action associated with modifying a compound to form another compound; and any other rule or any other action associated with modifying a compound to form a different compound.

A set of rule(s) and/or the one or more action(s) may also be selected that conform to any required or necessary structural, physical and/or chemical constraints associated with the compound, which ensure any modification(s) to the compound and/or subsequent modified compounds are actually feasible. The set of rule(s) and/or the one or more action(s) may further select from a set of relevant chemical groups comprising one or more of: one or more of atom(s); one or more molecule(s); one or more other compound(s); one or more bond(s); one or more functional group(s); and/or one or more chemically relevant aspects of the compound and the like. For example, a rule and/or action may include one or more action(s) for modifying a given compound in which the action(s) may include, by way of example only but is not limited to, addition/removal/change to the given compound of at least one or more of, by way of example only but are not limited to, a particular atom; particular molecule; other particular compound; bond; and/or functional group and the like. The one or more of particular atom(s); molecule(s); other compound(s); bond(s); and/or functional group(s) and the like may be selected from a set of chemically relevant atom(s); molecule(s); other compound(s) and/or compound fragment(s); bond(s); and/or functional group(s) and the like. These may be predefined and/or selected by an operator or automatically selected based on a knowledge base, or stored lists or one or more set(s) including data representative of the chemically relevant atom(s); molecule(s); other compound(s) and/or compound fragment(s); bond(s); and/or functional group(s) and the like for use in modifying the given compound or later compounds in respect of the desired propert(ies).

For example, a number of n>1 of rule(s)/actions(s) for modifying compound(s) may form a set of rules {Rᵢ}ᵢ₌₁ⁿ, where Rᵢ is the i-th rule/action for modifying a compound, which, by way of example only but not limited to, may include the following rule(s)/action(s):
R₁—adding a first chemical element to a compound;
R₂—adding a first compound fragment to a compound;
R₃—removing a first chemical element from a compound;
R₄—removing a first compound fragment from a compound;
R₅—adding a second chemical element to a compound;
R₆—adding a second compound fragment to the compound;
R₇—removing a second chemical element from a compound;
R₈—removing a second compound fragment from a compound;
. . .
Rᵢ—adding or reforming a bond between atoms of a compound;
R^{j}—breaking or removing a bond between atoms of a compound;
. . .
Rⁿ⁻¹—any other rule or any other action associated with modifying a compound to form another compound; and
Rₙ—any other rule or any other action associated with modifying a compound to form a different compound.

Each rule/action of the set of rules {Rᵢ}ᵢ₌₁ⁿ may be used one or more times to modify a compound from an initial compound to another compound. An ordered sequence of one or more rules (Rᵢ) may be selected from the set of rules, which may define how a first compound may be modified based on the ordered sequence of rules to form another compound. The sequence of rules is an ordered sequence to guarantee that the resulting compound can be derived from the first compound. It is not guaranteed that any ordering of the sequence of rules will result in the same resulting compound. For the purposes herein, a sequence of rules is considered to be an ordered sequence of rules which should be applied in a particular order to modify a first compound into the compound resulting from following the ordered sequence of rules when modifying the first compound.

The RL technique according to the invention as described herein may use one or more or a combination of ML techniques for generating one or more candidate compounds. An ML technique may comprise or represent one or more or a combination of computational methods that can be used to generate analytical models and algorithms that lend themselves to solving complex problems such as, by way of example only but is not limited to, prediction and analysis of complex processes and/or compounds. ML techniques can be used to generate compounds for use in the drug discovery, identification, and/or optimization in the informatics, chem(o)informatics and/or bioinformatics fields.

The ML technique(s) for generating candidate compounds from a set of desired properties and a starting compound may include, by way of example only but it not limited to, a least one ML technique or combination of ML technique(s) from the group of: a recurrent neural network/; convolutional neural network; reinforcement learning algorithm(s) based on neural networks; and any other neural network structure suitable for implementing the invention as described herein. Furthermore, one or more further neural network(s) may be applied or used for reading/receiving compound structures (or molecule structures and the like) for inputting these compound/molecule structures into the ML technique (e.g. RL technique) in a suitable format. Other ML techniques may be used to score compounds against the desired set of properties and/or estimate properties of compounds for scoring, which may be used to generate one or more, or a series of, ML models designed to predict one or more properties of compounds and output a property value or property score representing whether or how closely that compound exhibits or is associated with a property.

Further examples of ML technique(s) that may be used by the RL technique according to the invention as described herein may include or be based on, by way of example only but is not limited to, any ML technique or algorithm/method that can be trained or adapted to generate one or more candidate compounds based on, by way of example only but is not limited to, an initial compound, a list of desired property(ies) of the candidate compounds, and/or a set of rules for modifying compounds, which may include one or more supervised ML techniques, semi-supervised ML techniques, unsupervised ML techniques, linear and/or non-linear ML techniques, ML techniques associated with classification, ML techniques associated with regression and the like and/or combinations thereof. Some examples of ML techniques may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), deep NNs, deep learning, deep learning ANNs, inductive logic programming, support vector machines (SVMs), sparse dictionary learning, clustering, Bayesian networks, representation learning, similarity and metric learning, sparse dictionary learning, genetic algorithms, rule-based machine learning, learning classifier systems, and/or one or more combinations thereof and the like.

Some examples of supervised ML techniques may include or be based on, by way of example only but is not limited to, ANNs, DNNs, association rule learning algorithms, a priori algorithm, Éclat algorithm, case-based reasoning, Gaussian process regression, gene expression programming, group method of data handling (GMDH), inductive logic programming, instance-based learning, lazy learning, learning automata, learning vector quantization, logistic model tree, minimum message length (decision trees, decision graphs, etc.), nearest neighbour algorithm, analogical modelling, probably approximately correct learning (PAC) learning, ripple down rules, a knowledge acquisition methodology, symbolic machine learning algorithms, support vector machines, random forests, ensembles of classifiers, bootstrap aggregating (BAGGING), boosting (meta-algorithm), ordinal classification, information fuzzy networks (IFN), conditional random field, anova, quadratic classifiers, k-nearest neighbour, boosting, sprint, Bayesian networks, Naïve Bayes, hidden Markov models (HMMs), hierarchical hidden Markov model (HHMM), and any other ML technique or ML task capable of inferring a function or generating a model from labelled and/or unlabelled training data and the like.

Some examples of unsupervised ML techniques may include or be based on, by way of example only but is not limited to, expectation-maximization (EM) algorithm, vector quantization, generative topographic map, information bottleneck (IB) method and any other ML technique or ML task capable of inferring a function to describe hidden structure and/or generate a model from unlabelled data and/or by ignoring labels in labelled training datasets and the like. Some examples of semi-supervised ML techniques may include or be based on, by way of example only but is not limited to, one or more of active learning, generative models, low-density separation, graph-based methods, co-training, transduction or any other a ML technique, task, or class of unsupervised ML technique capable of making use of unlabeled datasets and/or labelled datasets for training and the like.

Some examples of artificial NN (ANN) ML techniques may include or be based on, by way of example only but is not limited to, one or more of artificial NNs, feedforward NNs, recursive NNs (RNNs), Convolutional NNs (CNNs), autoencoder NNs, extreme learning machines, logic learning machines, self-organizing maps, and other ANN ML technique or connectionist system/computing systems inspired by the biological neural networks that constitute animal brains. Some examples of deep learning ML technique may include or be based on, by way of example only but is not limited to, one or more of deep belief networks, deep Boltzmann machines, DNNs, deep CNNs, deep RNNs, hierarchical temporal memory, deep Boltzmann machine (DBM), stacked Auto-Encoders, and/or any other ML technique.

Although RL techniques may also be part of the class of ML techniques, it is to be appreciated by the skilled person that the RL technique(s) as described herein may use or apply any one or more suitable ML technique or combinations thereof and as mentioned/described above for generating one or more candidate compounds based on an initial compound (or subsequent candidate compound(s)) and desired property(ies) that the resulting candidate compound(s) is required to exhibit.

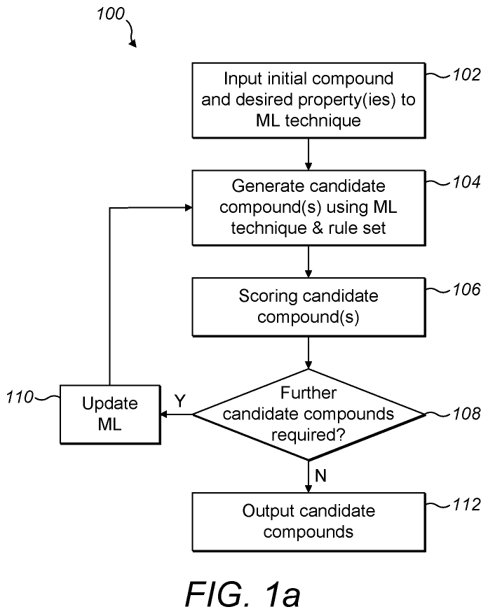

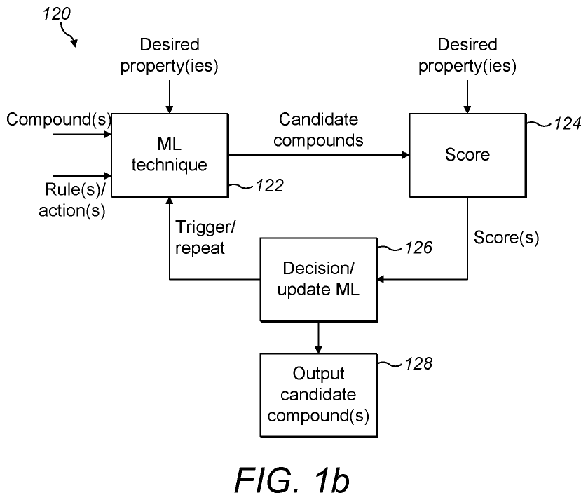

FIG. 1a is a flow diagram illustrating an example process 100 for designing a compound exhibiting one or more or a set of desired property(ies) according to the invention. The process 100 may use an ML technique and the set of desired property(ies) of a candidate compound to generate candidate compounds, which may be scored, and then used to, if necessary, adapt or update the ML technique in order for the ML technique to generate further more improved candidate compounds that may more exhibit the desired properties. The desired properties are predetermined or initially input to the process 100 and may include data representative of a list of characteristics or properties that a candidate compound is desired to exhibit. The steps of the process 100 may include one or more of the following steps:

In step 102, data representative of an initial compound (e.g. a starting compound or molecule or set of compound fragments) is input to the ML technique used to modify the initial compound and generate a candidate compound (or one or more candidate compounds). Data representative of the desired property(ies) of the candidate compounds is also input to the process, which may be used in the ML technique and/or scoring any candidate compound(s) generated. A set of rules for modifying compounds may also be input to the process 100 to allow the ML technique to select a sequence of rules or generate a sequence of actions used for modifying compounds to generate candidate compounds. The ML technique may also be configured to learn or be trained to model rules/actions for modifying compounds.

In step 104, the ML technique is used to generate a candidate compound or one or more candidate compound(s) by modifying a first compound (e.g. the initial compound and/or any subsequent candidate compound) based on the desired property(ies) and the set of rules for modifying compounds. The ML technique outputs one or more candidate compound(s).

In step 106, the candidate compound(s) are assessed and scored against the desired property(ies). One or more scores may be associated with each desired property. Each candidate compound may thus be scored against each desired property. The candidate compound may be simulated using atomistic computer simulations to determine a measure for each of the desired properties, which may be used to generate a score for each of the desired property(ies). The scores for each desired property may then be weighted and/or aggregated to generate an overall score indicating how close the candidate compound comes to exhibiting the desired property(ies).

In step 108, it is determined whether further candidate compound(s) are required based on the scoring of step 106. The determination may use previous scores from previous iterations of the process 100 to determine whether the score is a significant improvement on what candidate compound(s) have previously been generated by the ML technique. If the scoring indicates further compound(s) are required (e.g. ‘Y’) then the ML technique used to generate the candidate compounds may proceed to step 110, where the ML technique may be adapted or updated based on the scoring (e.g. the one or more scores associated with each desired property, or the weighted/aggregated score). The ML technique may be updated or adapted prior to starting another iteration of generating/modifying compounds. If it is determined that no further candidate compounds are required, e.g. it is determined that one or more candidate compound(s) exhibit the desired property(ies), then the process proceeds to step 112. The determination may further include one or more further decisions or an aggregate decision based on, by way of example only but not limited to, one or more of the following: whether a maximum number of iterations has occurred; whether there are no further significant improvements in the candidate compounds compared with previous iterations of candidate compounds; whether the scoring of each candidate compound plateaus compared with previous iterations; whether the scoring indicates one or more candidate compounds exhibit the desired properties; and/or there are no further significant improvements to the candidate compounds and it is evident these candidate compounds are the best ones that may be achieved.

In step 110, the ML technique is adapted or updated based on the scoring and/or the determination. The overall scoring for each candidate compound may be based on one or more scores associated with each desired property. The scoring indicates how well the candidate compound(s) match or fit to the desired property(ies). The RL technique initiates an updates or adaptation of the ML technique based on the score, which involves updating the parameters, coefficient(s) and/or weight(s) of the ML technique. The RL technique may penalise the ML technique based on, by way of example only but is not limited to, one or more of the following conditions: the scoring indicates the properties of the candidate compound are further away from the desired properties; the modified molecule/compound is too big/small; the modified molecule/compound exhibits any other undesirable quality or difference to the desired compound and/or the desired property(ies). On the other hand, the RL technique may reward the ML technique based on, by way of example only but not limited to, one or more of the following: the scoring indicates the modified molecule exhibits properties closer to the desired properties that are required.

Although the ML technique is adapted or updated after step 110, it is to be appreciated by the skilled person that the ML technique may be adapted or updated at anytime after scoring for the candidate compound in step 106 has been output or is known. In any event, the ML technique can be adapted or updated prior to repeating the generation step 104 of process 100. That is prior to performing another iteration of the generation of another one or more candidate compound(s). Furthermore, the update step 110 may include a further decision on whether the ML technique is required to be updated based on the scoring. For example, it may be advantageous to not immediately update the ML technique and allow it to perform another iteration or generation of another candidate compound. Instead of perturbing the ML technique by updating it to encourage it to generate a “better” candidate compound, the ML technique may be perturbed by allowing it to generation one or more further candidate compounds based on modifying the current one or more candidate compound(s). In addition, the update step 110 may further include a decision on whether the ML technique should be reverted to a previous version of the ML technique that may have generated a candidate compound that has a more improved score than the current candidate compound. The previous version of the ML technique may be used along with the corresponding previous candidate compound to perform another iteration or generation of further candidate compound(s) that may be assessed and/or scored in step 106 and/or a decision made in continuing the iterations/updating the ML technique in step 108.

After step 110, the RL technique performs another iteration of steps 104 and 106 of the design process 100. An iteration counter may be updated to keep track of the number of iterations that have been performed. In the next iteration, the process 100 proceeds to step 104 in which the ML technique, which may have been updated in step 110, is used to generate one or more candidate compound(s) starting from, by way of example only but is not limited to, either: a) the initial compound as input in step 102; or b) one of the candidate compound(s) of a previous iteration. The ML technique applies another sequence of rules/actions to modify the starting compound and output another modified candidate molecule/compound for scoring in step 106.

In step 112, it has been determined in step 108 that the process 100 should stop iterating and output data representative of one or more candidate compound(s). The data representative of each candidate compound may include, by way of example only but is not limited to, a description or representation of the candidate compound, the property scores and/or overall score given to the candidate compound, a sequence of rules/actions taken by the ML technique required to modify the initial candidate compound and end-up with the candidate compound; any other information that may be useful to an expert for assessing whether the output candidate compound is viable given how close it exhibits or is to the desired property(ies) input in step 102.

Furthermore, all generated candidate compounds and corresponding scores may be stored and, when it is decided to output one or more candidate compounds, the stored generated candidate compounds may be ranked based on their scores, and the topmost k ranked, k>=1, and data representative of the candidate compounds may be output. In addition to outputting the candidate compounds, data representative of the sequences of rules used to generate each candidate compound may be stored and also output to provide evidence of how the candidate compound may be composed and/or why it exhibits, or is close to exhibiting, the desired property(ies).

Inputting a compound to the ML technique in step 102 and/or step 104 may include, by way of example only but is not limited to, inputting: the initial compound; inputting a set of compound(s); inputting a set of compound fragments which may be combined or modified to produce one or more candidate compounds; inputting one or more candidate compound(s) generated from previous iterations of process 100; or any one or more or a combination of these. The ML technique may be configured to receive a compound or one or more compounds/fragments and generate a candidate compound or a set of candidate compounds based on the desired property(ies) and the set of rules and/or actions for modifying compounds.

Further modifications to process 100 may include, in step 108, determining whether to repeat the generating step 104 and/or update the ML technique in step 110 may be based on, by way of example only but is not limited to, the scoring indicating the candidate compound is closer to a compound that exhibits the desired property(ies); the scoring indicating the second compound exhibits the desired property(ies); whether a predetermined number of iterations of repeating the generating step 104 has been achieved or met; the second compound exhibiting at least one of more of the desired property(ies), whether any further improvements to the second compound are possible.

As described in step 106 of process 100, the candidate compound(s) are assessed and scored against the desired property(ies). The scores for each desired property may be based on, by way of example only but is not limited to, a certainty score, where one or more of the candidate compound(s) has a positive certainty score when those compounds substantially exhibit a majority or all of the desired property(ies). The certainty score may be a negative certainty score when one or more candidate compound(s) substantially do not exhibit some or even any of the one or more desired property(ies). Candidate compound(s) may also have a certainty score in between the positive certainty score and negative certainty score when those compounds substantially exhibit some or most of the one or more desired property(ies). The certainty score may be represented as a percentage certainty score, where the maximum positive certainty score is 100%, the minimum negative certainty score is 0%, and where the certainty score may be in between these extremes.

The assessment and scoring of a candidate compound being based on a desired set of property(ies) may include analysing the candidate compound against each of the desired property(ies), calculating a property value or score for each desired property in relation to the candidate compound, and/or calculating an aggregated score for the candidate compound based on the analysis. The scoring of the candidate compound may include calculating one or more property scoring metrics/values/scores based on, by way of example only but is not limited to, machine learning (ML) models of properties (e.g. solubility, toxicity, drug-target interaction, bioavailability, metabolism, and the like etc.), which may be configured to predict a property value/score of a compound in relation to a property, or calculate property values/scores of a candidate compound based on computer simulations and/or from laboratory experimentation or tests in a laboratory setting.

For example, the analysis may include performing on the candidate compound a computer simulation associated with one or more of the desired property(ies). Alternatively or additionally, the analysis may also include analysing the candidate compound using a knowledge based expert to determine whether the candidate compound exhibits or is closer to exhibiting one or more of the desired property(ies) or all of the desired property(ies). Alternatively of additionally, although this may be more costly and take longer, the analysis may include analysing the candidate compound using laboratory experimentation to determine whether the candidate compound exhibits or is closer to exhibiting one or more of the desired property(ies) or all of the desired property(ies) in the set of desired property(ies).

Alternatively or additionally, the assessment and/or scoring of the candidate compound may be based on one or more ML technique(s), each of which may have been trained on already known compounds or labelled training data for predicting whether a compound has a particular characteristic or property. Each trained ML technique may be associated with a different characteristic or property of a compound and may be configured to output a property value/score such as, by way of example only but not limited to, a probability, certainty score, or other classification or result that indicates whether the input compound has a particular characteristic or property that the ML technique has been trained to detect/determine.

Thus, the candidate compound may be input to one or more ML techniques associated with the set of desired properties, in which each ML technique outputs, for example, a property value/score such as a certainty score associated with a particular desired property to produce a set of probability/certainty scores, which may be used to determine whether the candidate compound is closer to exhibiting the desired properties or not compared with a previous iteration of process 100. Alternatively or additionally, the set of probability or certainty scores may be weighted and/or aggregated to provide an overall probability or certainty score that the candidate compound is closer to exhibiting the set of desired properties of not compared with a previous iteration process 100.

Although the scoring of the candidate compound is described as being implemented using, by way of example only but is not limited t, atomistic computer simulation and/or atomistic computer models that simulate or measure whether a compound as one or more property(ies), and/or using ML models trained for predicting whether a compound exhibits or is associated with a particular property, the skilled person would understand that the scoring of a candidate compound could be implemented using another ML technique that has been trained to score candidate compounds against a list of desired properties. Furthermore, the scoring ML technique may be based on RL techniques that may receive the candidate compounds, receive the desired properties and iteratively learn how to score candidate compounds over time.

Although a range of ML techniques have been described herein, it is preferred that the ML technique(s) that may be used may include, by way of example only but is not limited to, at least one ML technique or combination of ML technique(s) from the group of: a recurrent neural network (RNN) configured for predicting, starting from a first compound, a second compound exhibiting a set of desired property(ies); convolutional neural network (CNN) configured for predicting, starting from a first compound, a second compound exhibiting a set of desired property(ies); reinforcement learning (RL) algorithm configured for predicting, starting from a first compound, a second compound exhibiting a set of desired property(ies); and any other neural network (NN) structure configured for predicting, starting from a first compound, a second compound exhibiting a set of desired property(ies). Once the hidden layer structure of a NN has been determined, the weights, parameters and coefficients and/or may be updated and/or changed based on various update algorithms (e.g. back propagation etc.) based on the scoring used by the process 100.

FIG. 1b is a schematic diagram illustrating an example apparatus 120 for designing a compound exhibiting one or more desired property(ies) according to the invention. The apparatus 120 may include an iteration loop that includes a ML device 122, scoring device 124 and a decision/update device 126, which may be used to iteratively generate one or more sets of candidate compound(s). The output device 128 is used for outputting the candidate compound(s) and/or set(s) or sequence(s) of rule(s) for generating the output candidate compound(s) when the decision/update device 126 considers no further iterations are necessary or will yield an improved set of candidate compounds that may exhibit or be closer to exhibiting the desired property(ies).

The ML device 122 may be based on any suitable ML technique that may be used to generate a candidate compound based on an input compound or set of input compound(s), desired property(ies) and/or a set of rules for modifying the input compound(s). The ML device 122 may output a set of candidate compound(s) (e.g. one or more candidate compound(s)) to the scoring device 124. The scoring device 124 is configured to assess and/or score the set of candidate compound(s) based on the desired property(ies). A set of scores for each property and/or each candidate compound may be output, or a set of overall scores (or overall property scores based on the desired set of properties) for each candidate compound may be output from the scoring device 124. These candidate compound scores may be used to determine whether one or more of the candidate compound(s) are closer to exhibiting the desired property(ies) and whether the apparatus 120 should perform another iteration to generate further candidate compounds. The candidate compound score for each of the candidate compounds may be used to select those candidate compounds that are more likely to generate further candidate compounds that may be closer to exhibiting the desired property(ies).

The decision/update device 126 receives the score(s) of the candidate compounds from scoring device 124 and may use previous or historical performance/scoring data to determine whether the ML technique device 122 has improved in outputting a candidate compound that is closer to a compound exhibiting all of the desired property(ies), or will improve in future to output a candidate compound accordingly. The scores may also be used to trigger whether the ML technique device 122 updates or further adapts the ML technique, which may use the scores to adapt the associated weights, parameters, and/or coefficients and the like of the ML technique. The decision device 126 may use the score to reward or penalise the ML technique during the update, which will assist it in making different decisions or selections of rules when modifying further candidate compounds. The decision device 126 may further consider the ML technique needs to be reverted to a previous state, and so update the ML technique with the previous state of the ML technique. Once the decision device 126 decides to trigger a repeat of generating a candidate compound and, after updating/reverting the ML technique, the ML technique device 122 may be reconfigured to generate further candidate compounds based on, by way of example only but not limited to, the initial compound or set of compound(s), the current candidate compound(s), and/or one or more previous candidate compounds.

The decision/update device 126 may decide or determine that further iterations to generate further candidate compounds are not required and so output a candidate compound that has been found to be the best one or more candidate compounds that are closest to exhibiting the desired property(ies) based on the scores. That is, all generated candidate compounds may be stored and ranked based on their scores, and the topmost k ranked, k>=1, and data representative of the candidate compounds may be output. In addition to outputting the candidate compounds, data representative of the sequences of rules used to generate each candidate compound may be stored and also output to provide evidence of how the candidate compound may be composed and/or why it exhibits or is close to exhibiting the desired property(ies).

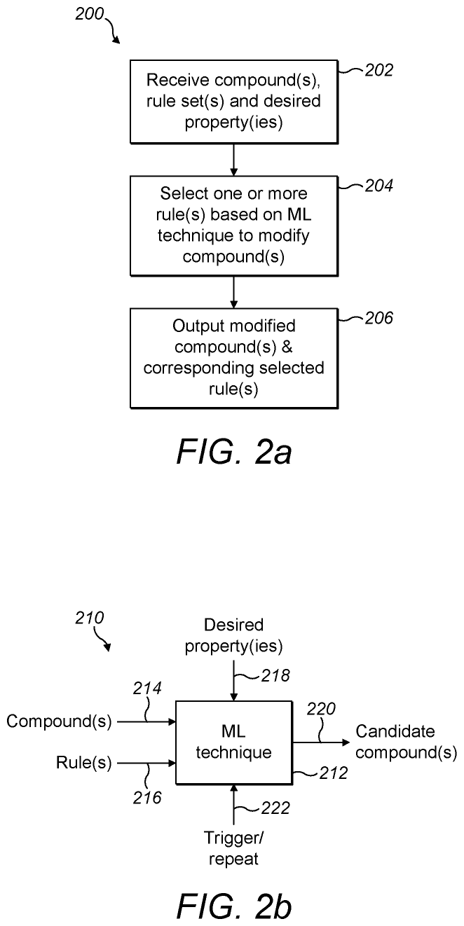

FIG. 2a is a flow diagram illustrating an example process 200 for an ML technique to follow when generating a candidate compound that may exhibit one or more desired property(ies) according to the invention. The example process 200 may be implemented in step 104 of the RL technique process 100, which may iteratively be used to generate one or more sets of candidate compounds. As described in steps 108 and 110 of process 100, the ML technique may be adapted and updated based on the scores of any output candidate compounds (e.g. rewarded and/or penalised). In this way, the ML technique “learns” to select improve sets or sequences of rules for modifying a compound to generate candidate compounds most likely to be closer to exhibiting the set of desired property(ies) required. The steps of the ML technique process 200 may be, by way of example only but not limited to, the following:

In step 202, the ML technique receives data representative of a set of one or more compound(s), data representative of a set of rule(s), which may include a set of action(s) for modifying compounds, and/or data representative of a desired set of property(ies) that a candidate compound may be modified to exhibit. In step 204, the ML technique may select one or more rules from the set of rules for modifying the set of one or more compound(s) and generate a set of one or more candidate compound(s). For example, the ML technique may receive a compound and then generate a first set of candidate compound(s), in which each candidate compound is based on the received compound being modified by a different rule in the set of rule(s). The ML technique may be configured to estimate, assess and/or score each of the candidate compounds in the set of candidate compounds based on the set of desired property(ies). This may assist the ML technique in selecting the best candidate compounds and the corresponding rules used to generate each best candidate compound from the first set of candidate compounds. That is, the ML technique may rank the set of candidate compounds and the corresponding rule(s) based on the estimate, assessment, and/or scoring, and then, generate a second set of candidate compounds based on the topmost ranked candidate compounds of the first set of candidate compounds and the corresponding set of rule(s). The ML technique may perform this iteration multiple times, or a set number of times before moving to step 206. In step 206, the ML technique may output a set of candidate compounds and/or corresponding set(s) of rule(s) for modifying the received compound(s). Each candidate compound that is output may have a corresponding selected set of rule(s) that can be used to generate the output candidate compound from the input compound, or even the initial compound input to the ML technique.

FIG. 2b is a schematic diagram illustrating an example apparatus 210 for an ML technique 212 that may be used in the iterative loop of apparatus 120 and/or process 100. The ML technique 212 receives data representative of one or more input compound(s) 214 and aims to generate a candidate compound or one or more candidate compound(s) 220 each of which may exhibit one or more desired property(ies) 218 or be closer to exhibiting the desired property(ies) 218 than the one or more input compound(s) 214. The ML technique 212 may be based on any ML technique or combination thereof as described herein. The ML technique 212 may receive at least data representative of a compound 214 or one or more compounds (or compound fragments), which the ML technique 212 may use as a basis along with a set of rules 216 for modifying compounds to generate a set of candidate compounds 220 that are more likely to exhibit the desired property(ies) 218 than the input compound.

However, it is noted that in the early iterations of the apparatus 120, the ML technique 212 may initially produce candidate compounds that might be less likely to exhibit the desired property(ies) 218. That is, the candidate compounds may perform poorly when scoring the candidate compounds, so based on the scoring, the ML technique 212 may receive a trigger or update trigger and scores that are used to penalise the ML technique 212 during its update/adaptation. This is used to teach the ML technique 212 not to select those sequence(s) of rule(s); or initially select a portion of the set of rule(s) for modifying compounds; and/or to make the decisions that produced such poorly performing candidate compounds. Thus, the apparatus 200, becomes part of a RL technique which is updated/adapted and rewarded or penalised depending its output candidate compounds. Over time or many iterations of an RL iterative loop of apparatus 100, the ML technique 212 of apparatus 200 may be trained to select one or more sequence(s) of rules that can be used to generate candidate compounds that are closer to exhibiting the desired property(ies), or that actually exhibit all of the desired property(ies) with a high degree of certainty or at a high level.

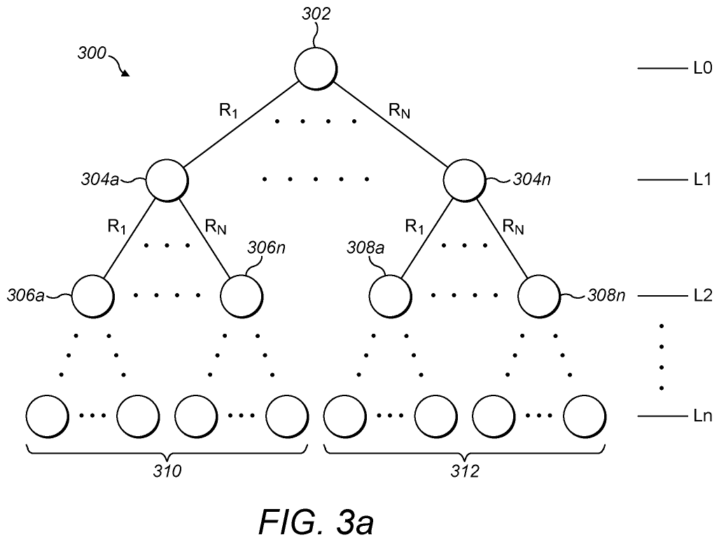

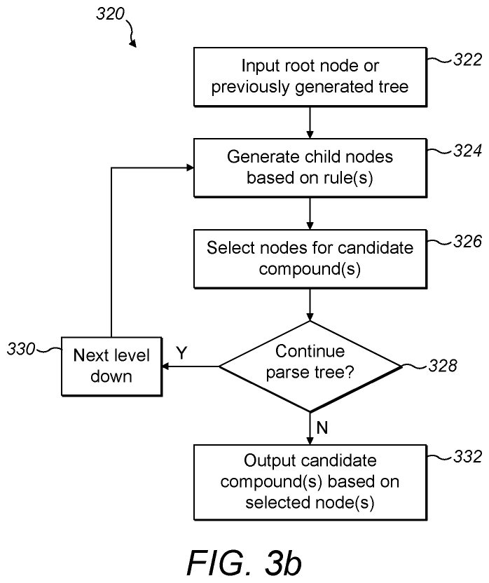

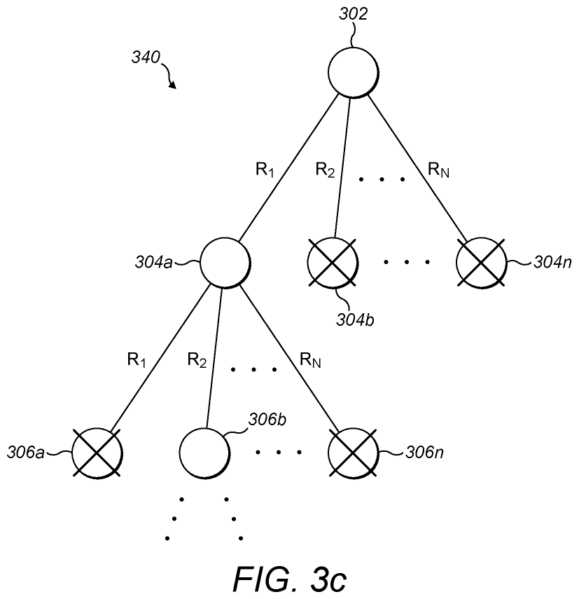

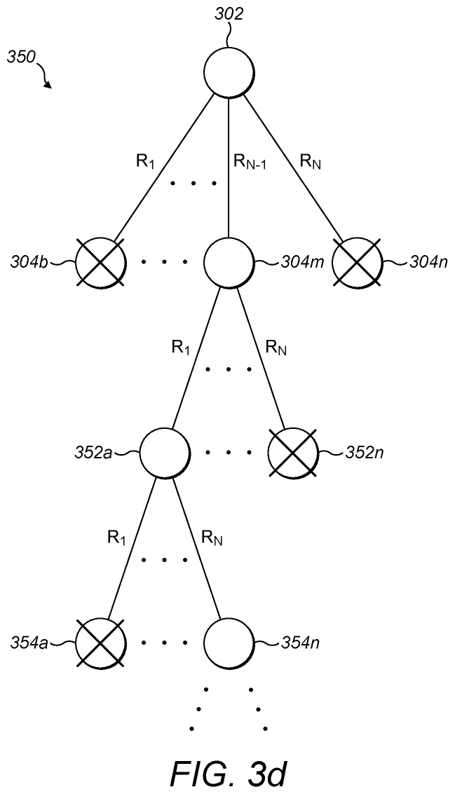

FIG. 3a is a schematic diagram illustrating an example a tree-based data structure 300 for use by an ML technique to generate a compound exhibiting one or more desired property(ies) according to the invention. The ML technique 212 of apparatus 200 may make use of a tree-based data structure 300 that may include a plurality of nodes 302, 304a-304n, 306a-306n, 310-312 and a plurality of edges R₁, . . . , Rₙ. The plurality of nodes includes a root node 302 representing a compound from which a plurality of edges R₁, . . . , Rₙ connect to a set of child nodes 304a-304n. The plurality of edges R₁, . . . , Rₙ represent the set of rule(s) for modifying a compound. There is one edge for each rule in the set of rule(s). Thus, child nodes 304a-304n are connected to each edge R₁, . . . , Rₙ from the root node 302, where each child node 304a-304n represents the compound of the root node 302 modified by the corresponding rule R₁, . . . , Rₙ representing the connecting edge.

The tree 300 may be generated down to a number of m levels, in which each node at each level uses the same set of edges R₁, . . . , Rₙ. That is, each of the edges R₁, . . . , Rₙ connects a parent node 302 to a child node 304a-304n, where a parent node 302 represents a compound and each edge R₁, . . . , Rₙ from a parent node 302 to a child node 304a-304n represents a rule (or an action) of the set of rules R₁, . . . , Rₙ (or a plurality of actions) performed on the compound of the parent node 302 that results in the compound of the child node 304a-304n. The root node 302 of the tree 300 is the first compound that may be input to the ML technique and subsequent nodes 304a-304n, 306a-306n, 308a-308n, 310, and 312 correspond to one or more sets of candidate compound(s) or a plurality of candidate compound(s). Given that each node in the tree 300 uses the same set of edges R₁, . . . , Rₙ, then following a path along the edges down the tree to a particular node at the m-th level (e.g. Lₘ) will give a sequence of rules that can be used to modify the first compound into a compound associated with the particular node at the m-th level. Although parsing a whole tree 300 based on the rule set along all the rule edges may yield all possible candidate compounds and the corresponding sequences of rule(s) for each compound, this leads to a ML technique with exponential complexity. For example, if there are a number of n rules in the set of rules for modifying compounds, then the m-th level of a full tree 300 for m>=0, would have a number of n^{m} nodes or candidate compounds.

Thus, rather than generate an entire tree 300 to the m-th level, the ML technique may expand the tree data structure based on assessing or scoring each of the one or more nodes corresponding to the set of candidate compound(s). Alternatively, a tree search on the tree data structure 300 may be performed to generate a set of candidate compounds based on only those sequence(s) of rules that yield the candidate compounds with the best scores or that are closer to the set of desired property(ies).

For example, a simple ML technique may be configured to maintain a tree data structure 300 and only increase it one level at a time on each iteration of process 100. Thus, a first compound is input as the root node 302 of the tree and in the first iteration of process 100, the ML technique may output, in step 104, a set of candidate compounds based on the set of child nodes 304a-304n. In step 106, the process 100 may assess the set of candidate compounds and output a corresponding set of scores. In step 108, a decision may be made to perform another iteration as there are too many candidate compounds and so the ML technique may be updated based on the set of scores. For example, those child nodes 304a-304n that have a score above a certain threshold may be retained, whilst those child nodes 304a-304n that have a score below the certain threshold may be pruned, or removed. Thus, in the next iteration, the ML technique may maintain and/or grow the tree-based structure 300 based on only those nodes 304a-304n that have been retained and the set of rules in each iteration. However, given that the set of rules R₁, . . . , Rₙ may be very large (e.g. greater than 1000s or greater than 1,000,000s), this may still yield a very large set of candidate compounds from which to select in each iteration. Thus, the ML technique may be configured to estimate, assess and or select only those rule edges R₁, . . . , Rₙ and/or child nodes at each level of the tree data structure 300 that are more likely to yield suitable candidate compounds.

FIG. 3b is a flow diagram of an example process 320 for implementing a tree-based ML technique according to the invention. This process 320 may be implemented by a ML technique in each iteration of process 100, where the ML technique is configured to maintain a tree-based data structure. The process may include the following steps: in step 322, a first compound may be input as the root node, or a previously generated tree may be input in which the leaf nodes each represent a set of candidate compounds. In step 324, child nodes of the current root node and/or parent nodes (or leaf nodes) are generated based on the set of rules R₁, . . . , Rₙ and also based on the ML technique, which may be configured to select a subset of the set of rules R₁, . . . , Rₙ for generating the child nodes. In step 326, the ML technique may be further configured to select a set of the newly created child nodes as a set of candidate compounds. The selection may be configured and based on how the ML technique is implemented and also on whether or how it is updated in each iteration of process 100. In step 328, it is determined whether to continue to parse the tree data structure and move down to the next level in the tree and generate further children nodes, or to output the selected set of child nodes as a selected set of candidate compounds. If it is determined to parse the tree data structure (e.g. ‘Y’), then in step 330 the next level of the tree data structure is generated and step 324 is performed to generate further child nodes for that level based on the set of rule(s) and the ML technique. If it is determined to output a set of candidate compounds (e.g. ‘N’), then, in step 332, the ML technique outputs candidate compound(s) based on the selected set of child nodes. The ML technique may also output the corresponding sequence(s) of rules, which can be generated by following a path from the root node to each of the selected child node(s).

It is noted that the ML technique may be updated in each iteration of process 100, and the ML technique may be triggered to, by way of example only but is not limited to, restart the generation and maintenance of the tree data structure starting from the first compound as a root node and using the updated ML technique to iteratively generate the child nodes and parse the tree data structure up to a number of levels or iterations, select and output a set of child nodes as candidate compounds; and/or start from the current set of candidates or the currently parsed tree data structure and continue to generate child nodes based on the updated ML technique; or start from a previous set of candidate nodes and so use a previously parsed tree data structure and continue parsing the previous tree data structure based on the updated ML technique, which should generate further different subsets of child nodes.

FIGS. 3c and 3d are schematic diagrams of example tree data structures 340 and 350 that have been parsed based on the process 320 of FIG. 3b to generate a set of one or more candidate compounds that may be closer to exhibiting the desired property(ies) according to the invention. It is assumed that the process 100 uses a tree-based ML technique based on the process 320. The tree-based ML technique receives a compound and a set of rules R₁, . . . , Rₙ. In the first iteration of process 100, the ML technique builds a tree 340 starting from the root node 302, which represents the initial compound. The ML technique then generates one or more child nodes 304a-304n based on expanding the root node 302 based on the set of rules R₁, . . . , Rₙ. The structure of the ML technique may be configured to select or assess which child nodes 304a-304n may be suitable or retained as candidate compounds. As can be seen in FIG. 3c, the tree-based ML technique, for some reason due to the current structure/weights/parameters or other data representing the underlying ML technique, only retains child node 304a as a candidate compound and discards child nodes 304b-304n.

The ML technique may decide (e.g. in step 328 of process 320) to generate a further set of child nodes based on the retained child node 304a. In this case, the structure of the ML technique may be configured to select or assess which child nodes 306a-306n may be suitable or retained as further candidate compounds. As can be seen in FIG. 3c, the tree-based ML technique, for some reason, only retains child node 306b as a candidate compound and discards child nodes 306a and 306c-306n. At this point, the ML technique may decide (e.g. in step 328 of process 320) to output the final set of child nodes as a set of candidate compounds and moves to step 332 to output the child node 306b as the candidate compound. This candidate compound was generated from the first compound (e.g. root node 302) based on applying the sequence of rules R₁ and R₂ to the first compound. That is, applying R₁ to the first compound represented by root node 302 generates the compound represented by child node 304a, applying the next rule R₂ in the sequence to the compound represented by the child node 304a generates the compound represented by child node 306b. Thus, a candidate compound based on the sequence of rules {R₁, R₂} may be output by the ML technique.

In step 106 of process 100, the candidate compound that is output by the tree-based ML technique may be assessed and scored. Given this is the first iteration of process 100, it is most likely that step 108 decides that further candidate compounds are required. In step 110, the current structure/weights/parameters or other data representing the underlying tree-based ML technique may be updated based on the scoring of the output candidate compound using suitable update algorithms (e.g. if the underlying ML technique is NN based, then a weight update based on backpropagation techniques might be used). Given the ML technique has been updated, in this example, the ML technique starts to rebuild the tree based on the initial starting compound. This is because, given the update to the ML technique, the ML technique may make different decisions/selections of the child nodes to possibly yield an improved candidate compound.

Referring now to FIG. 3d, after updating the tree-based ML technique in step 104, the updated ML technique receives a compound (the original compound) and the set of rules R₁, . . . , Rₙ. In the second iteration of process 100, the ML technique rebuilds a tree 350 starting from the root node 302, which again represents the initial compound. The ML technique then generates one or more child nodes 304a-304m and 304n based on expanding the root node 302 based on the set of rules R₁, . . . , Rₙ. The structure of the updated ML technique may have been configured to select or assess which child nodes 304a-304m and 304n may be suitable or retained as candidate compounds. As can be seen in FIG. 3d, the updated tree-based ML technique, for some reason due to the new updated structure/weights/parameters or other data representing the underlying ML technique, only retains child node 304m as a candidate compound and discards the other child nodes 304a-304n other than child node 304m.

The updated ML technique may further decide (e.g. in step 328 of process 320) to generate a further set of child nodes 352a-352n based on the retained child node 304m and the set of rules R₁, . . . , Rₙ. In this case, the structure of the ML technique may be configured to select or assess which child nodes 352a-352n may be suitable or retained as further candidate compounds. As can be seen in FIG. 3d, the tree-based ML technique, for some reason, only retains child node 352a as a candidate compound and discards child nodes 352b-352n. At this point, the ML technique may decide (e.g. in step 328 of process 320) to output the final set of child nodes as a set of candidate compounds or not. In this case, the ML technique continues to generate another set of child nodes 354a-354n based on child node 352a and set of rules R₁, . . . , Rₙ. In this case, the structure of the ML technique may be configured to select or assess which child nodes 354a-354n may be suitable or retained as further candidate compounds. As can be seen in FIG. 3d, the tree-based ML technique, for some reason, only retains child node 354n as a candidate compound and discards child nodes 354a-354m. At this point, the ML technique may decide (e.g. in step 328 of process 320) to output the final set of child nodes as a set of candidate compounds.

In this example, the process 320 moves to step 332 to output the child node 354n as the candidate compound. This candidate compound was generated from the first compound (e.g. root node 302) based on applying the sequence of rules Rₙ₋₁, R₁, and Rₙ in this particular order to the first compound. That is, applying Rₙ₋₁ to the first compound represented by root node 302 generates the compound represented by child node 304m, applying the next rule R₁ in the sequence to the compound represented by the child node 304m generates the compound represented by child node 352a, and applying the next rule Rₙ in the sequence to the compound represented by the child node 352a generates the compound represented by child node 354n. Thus, a candidate compound based on the sequence of rules (Rₙ₋₁, R₁, Rₙ) may be output by the ML technique.

In step 106 of process 100, the candidate compound based on the sequence of rules (Rₙ₋₁, R₁, Rₙ) that is output by the tree-based ML technique may be assessed and scored. Given this is the second iteration of process 100, it is most likely that step 108 decides that further candidate compounds are required. In step 110, the current structure/weights/parameters or other data representing the underlying tree-based ML technique may be updated based on the scoring of the output candidate compound using suitable update algorithms (e.g. if the underlying ML technique is NN based, then a weight update based on backpropagation techniques might be used). Given the ML technique has been updated or based on the scoring, and whether the scoring of the candidate compound in this iteration of process 100 is better than the previous scoring, then the ML technique may start to rebuild the tree based on the initial starting compound; or if the candidate compound is much closer to a compound that exhibits the desired property(ies), the current tree-based structure may be used to further parse or generate further child nodes on the tree 350. In any event, given the update to the ML technique, the ML technique should make different decisions/selections of the child nodes going forward and may yield further improved candidate compounds. Both processes 100 and 320 may be further iterated with current or rebuilt trees and starting compounds as described herein.

Although tree-based data structures may be used to store and/or generate a set of candidate compounds, alternative examples compound modification strategies based on one or more ML technique may also be applied. For example, an ML technique may be used that generates an N-dimensional action/rule space based on the set of rules, where Nis large enough such that each rule/action in the set of rules/actions may be represented by a unique N-dimensional vector in the N-dimensional vector space (e.g. N>>2). The elements of each N-dimensional vector may be, by way of example only but are not limited to, real and/or continuous values. Example ML technique capable of learning and generating an N-dimensional vector space based on the set of rules/actions that may include, by way of example only but are not limited to, neural network based structures. For example, NN structures may use one or more hidden layers of hidden units or cells that can be trained to generate an N-dimensional vector space for the set of rule(s)/action(s), which may then be used to select appropriate rule(s) for modifying compounds.

A set of rules/actions for modifying compounds has a large number of possible rules/actions that may be made for modifying compounds. For example, there may be 1000s or 1,000,000s of possibilities of different rules/actions in the set of rules for modifying compounds. Although the tree-based data structure may be useful, it is limited when the number of rules/actions greatly increases and may become more inefficient. Instead, a ML technique that encodes or maps the rule set into an N-dimensional space, a so-called N-dimensional rule/action vector space, might assist in selecting the sequence of rules for modifying a compound to generate a suitable set of candidate compound(s) that may exhibit the desired property(ies). A compound could be mapped into the N-dimensional rule/action vector space and then the closest or approximately closest rule/action vectors to the mapped compound may be selected for modifying the compound.

This encoding/mapping may be performed by numerous ML techniques such as, by way of example only but is not limited to, neural network structures and the like. Neural network structures typically use hidden layers that may be configured to encode/map a set of rules into an N-dimensional space, where each rule of the set of rule(s) being encoded or represented as a rule/action vector in the N-dimensional vector space. The neural network may also be configured to map a starting compound into the N-dimensional rule/action space as a compound proto-rule/action vector. The ML technique may then search or determine one or more closest rule/action vectors to the compound proto-rule/action vector. The rules/actions associated with the determined one or more closest rule/action vectors may then be used to each modify the compound to generate one or more corresponding candidate compounds.

For example, a nearest neighbour search algorithm may be used to determine the one or more closest rule/action vectors to the mapped compound in the N-dimensional vector space, the so-called N-dimensional rule/action vector space. There are many nearest neighbour algorithms that may be applicable such as, by way of example only but not limited to, k-nearest neighbour algorithm, approximate nearest neighbour algorithm, all nearest neighbour algorithms and the like. For simplicity, it is assumed that the encoding ML technique may be configured to solve a k-nearest neighbour search problem and find the k-nearest neighbour rule/action points that are closest to the mapped proto-action/rule in the N-dimensional space, where k>=1. If k>1, then multiple candidate compounds may be generated. Once the k-nearest set of action/rule(s) have been identified in the N-dimensional space, they may be decoded or demapped to the corresponding action(s)/rule(s) of the set of rules and applied to the corresponding compound for modifying the compound and generating one or more candidate compounds.

Thus, the encoding ML technique may encode all compounds and encode all the actions/rules that may be performed on the compounds into an N-dimensional space and determine the k-nearest neighbouring actions/rules to the compound. The compounds and actions/rules are thus located in the same N-dimensional space. This N-dimensional space defines all the possible rules/actions that could be taken on a compound. By mapping the compound also into the N-dimensional space as a proto-rule/action, or a so-called compound proto-rule/action vector, it may be possible to predict the most likely rule/action that should be taken to modify the compound into a candidate compound that may be closer to a compound exhibiting the desired property(ies). For example, the nearest neighbour rule/action vectors in the N-dimensional space that are closest to the compound proto-action/rule vector may be used to generate a set of candidate compounds.

Applying this encoding ML technique to the RL technique of process 100 may allow the encoding ML technique to adjust the N-dimensional space such that it learns to select a sequence of rules from the set of rules that may generate a set of one or more candidate compounds that may be closer to, or to exhibit all of, the desired property(ies). Initially, the NN of the ML technique may be uninitialised and know nothing about the possible rules/actions that should be applied to a compound. Instead, the NN may simply generate an N-dimensional space based on the set of rule(s) and compounds, then as the process 100 iterates, the NN of the ML technique may be updated based on the scoring (e.g. in steps 106-110). The scoring is an indication of how close one or more candidate compounds are to a compound that exhibits the desired property(ies) and can be used to update the NN and hence refine the N-dimensional vector space.

That is, the NN may be rewarded if it generates candidate compounds closer to a compound exhibiting the desired property(ies) or it is penalised when it generates candidate compounds that exhibit less than the desired property(ies). As the NN (or encoding ML technique) is updated based on the scoring of process 100, it will refines the N-dimensional space and thus the rule/action points and proto-rule/actions that define candidate compounds closer to the desired property(ies) will move closer together in the N-dimensional space.

As the NN is updated by process 100 based on the scoring, which is based on whether the candidate compounds are closer to the desired property(ies), then the NN or the N-dimensional space represented by the NN reflects how close to the desired properties the candidate compounds are getting and possibly what the final candidate compound may look like.

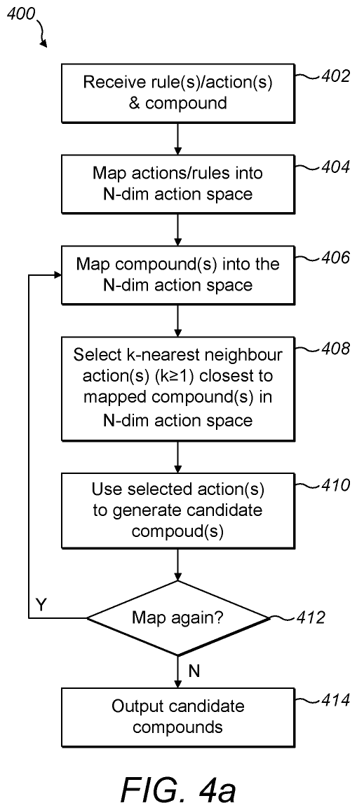

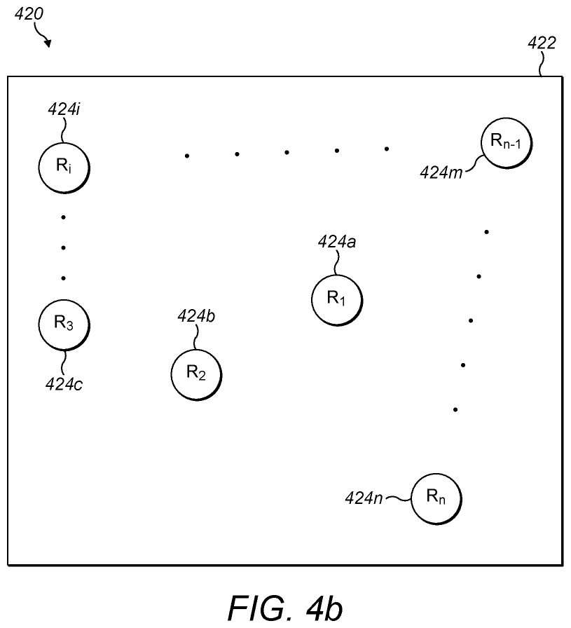

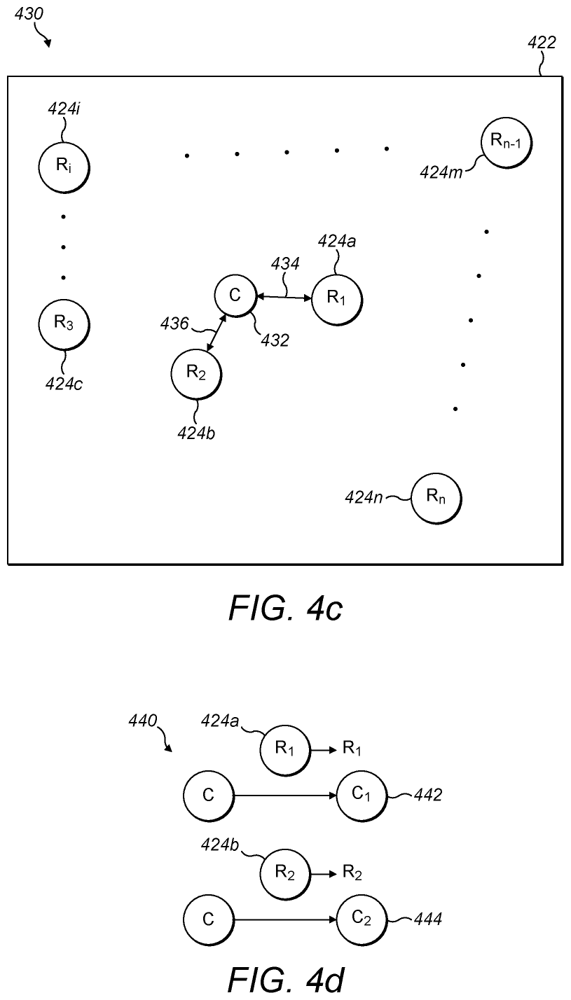

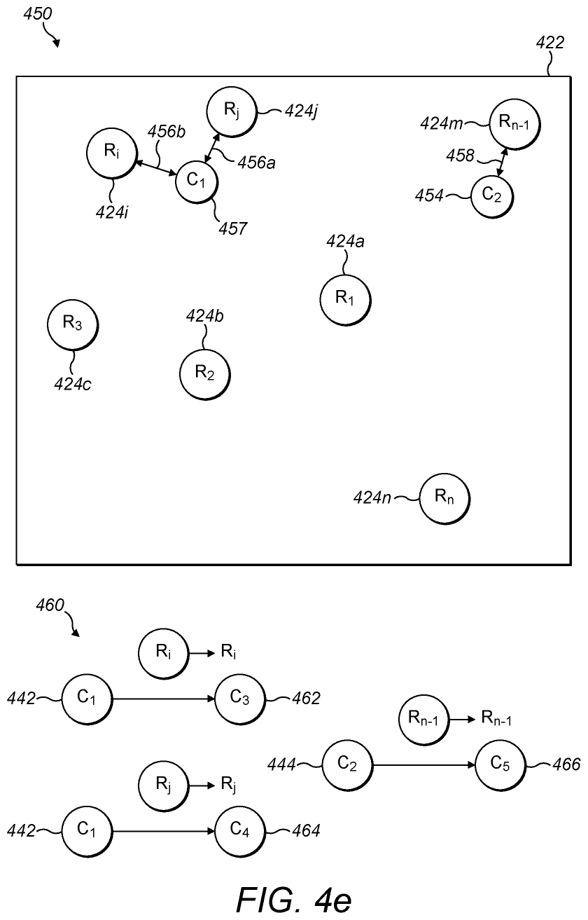

FIG. 4a is a flow diagram illustrating an example encoding process 400 for a encoding-space based ML technique to generate a compound exhibiting one or more desired property(ies) according to the invention. This process 400 may also be implemented by a ML technique in each iteration of process 100, where the ML technique is configured to maintain an N-dimensional rule/action space and adapt the N-dimensional space based on the scoring of candidate compounds.

The process 400 may include the following steps: in step 402, the set of rules and/or actions for modifying compounds is received, these may be mapped into the N-dimensional rule/action space; the process also receives data representative of the first or starting compound(s)/compound fragment(s) from which the set of one or more candidate compounds can be generated. In step 404, the set of rules/actions are encoded or mapped into the N-dimensional space. In step 406, the starting compound or fragments and/or, if this is another pass of process 400 and/or process 100, one or more candidate compound(s) may be encoded and/or mapped into the N-dimensional space. For example, the ML technique may implement a NN in which the hidden layers define the N-dimensional space into which the set of rules/actions and/or compounds are encoded or mapped.

In step 408, a subset of the rules/actions mapped in the N-dimensional action space may be selected that are nearest neighbours to the compound(s) when mapped in the N-dimensional rule/action space are selected. For example, the k-nearest neighbour rules/actions to a compound in the N-dimensional space may be selected. Given there may be more than one compound mapped in the N-dimensional rule/action space, each mapped compound in the N-dimensional space may have a subset of the k-nearest neighbour rules/actions that have been mapped in the N-dimensional space. Thus, one or more subsets of rules/actions that are the k-nearest neighbour rule/actions to corresponding one or more compounds mapped in the N-dimensional space may be selected. That is, a set of k rules/actions mapped in N-dimensional space that are considered the closest, based on a distance function or similarity function, to the compound mapped in N-dimensional space are selected, where k>=1.

In step 410, each subset of rules/actions in the N-dimensional space may be applied to the corresponding compound to generate one or more sets of candidate compound(s). For example, the subset of rules/actions mapped in N-dimensional space may be decoded into the corresponding rules/actions of the set of rules, and then may be applied to the corresponding compound, or used to modify the corresponding compound accordingly. In step 412, the process 400 may determine whether to perform one or more iteration(s) of the mapping, selecting and modifying steps 406, 408 and 410. If it is determined to perform more than one iteration (e.g. ‘Y’), then step 412 proceeds to step 406 for mapping/encoding each of the sets of one or more candidate compounds into the N-dimensional space. In step 412, if it is determined to not perform a further iteration of the mapping, selecting and modifying steps 406, 408 and 410 (e.g. ‘N’), then the process 400 proceeds to step 414 for outputting data representative of the one or more sets of candidate compounds and/or corresponding subset of rules/actions that can be used to modify the starting compound to generate the one or more sets of candidate compounds.

The encoding ML technique may be implemented in step 104 of process 100 and configured to output a set of candidate compounds, which may be one or more candidate compounds, from step 104 to the scoring step 106 of process 100. Step 108 may determine whether further iterations of process steps 110, 104 and/or 106. Steps 108 or 110 may determine whether it is necessary to update or further adapt the encoding ML technique implementing process 400 based on the scoring in step 110. Adapting or updating the encoding ML technique based on the scoring further refines the N-dimensional vector space to better describe the locations of the rule(s)/action(s) that are more suitable for generating/modifying compounds that are closer to, or that exhibit, the desired property(ies).

FIGS. 4b-4e are a schematic diagrams illustrating example states 420, 430 and 450 of an N-dimensional action/rule space 422 and how this may be used by an encoder-based ML technique that implements process 400 to generate a set of candidate compound(s) that are more likely to exhibit desired property(ies) according to the invention. As described with above, the N-dimensional vector space 422 is based on the set of rules/actions for modifying compounds and may be created, based on the structure of the encoder-based ML technique, in which each of the different rules/actions in the set of rules/actions can be encoded or mapped to an unique N-dimensional vector in the N-dimensional vector space 422.

As described previously, a rule for modifying a compound may comprise or represent data representative of any principle, operation, regulation, procedure, action or any other command, code or instruction or data format that may be used to describe modifying a compound from a first compound to a second compound. Thus, a set of rules for modifying compounds may comprise or represent data representative of one or more rules for modifying compounds or a plurality of rules for modifying compounds.

For example, as previously described, a number of n>1 of rule(s)/actions(s) for modifying compound(s) may form a set of rules {Rᵢ}ᵢ₌₁ⁿ, where Rᵢ is the i-th rule/action for modifying a compound, which, by way of example only but not limited to, may include the following rule(s)/action(s):
R₁—adding a first chemical element to a compound;
R₂—adding a first compound fragment to a compound;
R₃—removing a first chemical element from a compound;
R₄—removing a first compound fragment from a compound;
R₅—adding a second chemical element to a compound;
R₆—adding a second compound fragment to the compound;
R₇—removing a second chemical element from a compound;
R₈—removing a second compound fragment from a compound;
. . .
Rᵢ—adding or reforming a bond between atoms of a compound;
Rⱼ—breaking or removing a bond between atoms of a compound;
. . .
Rⁿ⁻¹—any other rule or any other action associated with modifying a compound to form another compound; and
Rₙ—any other rule or any other action associated with modifying a compound to form a different compound.

Each rule/action of the set of rules {Rᵢ}ᵢ₌₁ⁿ may be used one or more times to modify a compound from an initial compound to another compound. An ordered sequence of one or more rules (Rᵢ) may be selected from the set of rules, which may define how a first compound may be modified based on the ordered sequence of rules to form another compound.

Referring to FIG. 4b, an example representation of a first state 420 of the N-dimensional vector space 422 is illustrated in which the encoding ML technique may encode or map the set of rules {Rᵢ}ᵢ₌₁ⁿ for modifying compounds to unique N-dimensional vectors in the N-dimensional vector space 422. The plurality of rules/actions of the set of rules/actions are mapped to the N-dimensional vector space 42 in any suitable manner or using any one or more ML technique(s). For example, a ML technique based on neural network structure may be used to encode and/or map the plurality of rules/actions to the N-dimensional vector space 422, so-called N-dimensional rule/action space 422, as defined by the neural network structure.

In step 404 of process 400, the encoding ML technique maps each of the rules/actions in the set of rules {Rᵢ}ᵢ₌₁ⁿ into the N-dimensional rule/action space 422. This is illustrated, for example, in FIG. 4b in which the rule/action represented by R₁ may be mapped to N-dimensional vector action/rule 424a, the rule/action represented by R₂ may be mapped to N-dimensional vector action/rule 424b, the rule/action represented by R₃ may be mapped to N-dimensional vector action/rule 424c, and so on, the rule/action represented by Rᵢ may be mapped to N-dimensional vector action/rule 424i, and so on, the rule/action represented by Rₙ₋₁ may be mapped to N-dimensional vector action/rule 424m, and the rule/action represented by Rₙ may be mapped to N-dimensional vector action/rule 424n.

FIG. 4c, illustrates an example second state 430 of the N-dimensional rule/action space 422 in which the encoding ML technique maps a compound (e.g. represented by the letter ‘C’) into the N-dimensional rule/action space 422, e.g. step 406 of process 400. The compound C is represented as a compound proto-rule/action vector 432. Given that the compound C has been mapped into the N-dimensional rule/action space 422 as the proto-rule/action vector 432, it is now possible to select one or more rule/action vectors 424a-242n that may be near the location of the compound C, i.e. proto-rule/action vector 432, when mapped in the N-dimensional rule/action space 422.

In step 408 of process 400, once the compound C is mapped as a compound proto-rule/action vector 432 in the N-dimensional rule/action space 422, the one or more closest rule/action vectors to the compound proto-rule/action vector 432 may be selected. For example, a metric or distance metric/criterion such as, by way of example only but is not limited to, Euclidean distance between N-dimensional vectors, or any other metric or criterion useful for estimating the closest rule/action vectors to the compound proto-rule/action vector in the N-dimensional rule/action space may be used to estimate the k-nearest neighbour vectors to the compound proto-rule/action vector 432. In this example, a distance metric 434 between action/rule 424a and compound proto-rule/action vector 432 is determined, and a distance metric 434 between action/rule 424b and compound proto-rule/action vector 432 are determined to be the smallest distance metrics amongst the n-distance metrics. In this example, it is assumed for simplicity that k=2 and so 2 action/rule vectors are selected to be the subset of action/rule vectors that are closest to compound proto-rule/action vector 432. Although k=2 in this example, this is by way of example only and the patent is not so limited, it is to be appreciated by the skilled person that that k>=1 and that any value may be chosen for k as long as it is greater than or equal to 1. In this example, action/rule 424a and action/rule 424b are selected as the subset of rule/action vectors that may be used to modify compound C.

FIG. 4d is a schematic diagram illustrating an example synthesis 440 (e.g. modification) of compound C into compounds C₁ 442 and C₂ 444 using the selected subset of action/rule vectors 424a and 424b (e.g. step 408 of process 400). The selected subset of action/rule vectors 424a and 424b may be demapped or decoded to a subset of rules/actions including the actions/rules R₁ and R₂, respectively. Then, each of these rules are used to separately modify compound C into compounds C₁ 442 and C₂ 444 by applying these rules/actions to compound C. For example, compound C₁ 442 would be the compound that results from modifying compound C with R₁, such as adding a first chemical element to the compound C to form compound C₁ 442. Compound C₂ 444 would be the compound that results from modifying compound C with R₂, such as adding a first compound fragment to the compound C to form compound C₂ 444.

FIG. 4e is a schematic illustration of another example state 450 in which the compounds C₁ 442 and C₂ 444 are mapped into the N-dimensional action/rule space 422. For example, in step 412 of process 400, it may be decided perform multiple iterations of generating candidate compounds by mapping the previously generated candidate compounds into the N-dimensional space to find further rules/actions for adding to the subset of rules/actions. The encoding ML technique maps candidate compounds C₁ 442 and C₂ 444 into the N-dimensional rule/action space 422, e.g. step 406 of process 400. The compounds C₁ 442 and C₂ 444 are represented as compound proto-rule/action vectors 452 and 454, respectively. Once mapped, further rule/actions may be selected by finding the k-nearest neighbour action/rule vectors 424a-424n to compound proto-rule/action vectors 452 and 454, which represent compounds C₁ 442 and C₂ 444 in the N-dimensional action/rule space 422.

In this example, a distance metric 456a between action/rule 424j and compound proto-rule/action vector 452 is determined, and a distance metric 456b between action/rule 424i and compound proto-rule/action vector 452 is determined to be the two smallest distance metrics amongst the n-distance metrics to compound proto-rule/action vector 452 (e.g. k=2 in this example). Action/rule 424i and action/rule 424j are selected to be included into a subset of rule/action vectors for further separately modifying C₁ 442. Action/rule 424m is selected to be included into another subset of rule/action vectors for further separately modifying C₂ 444.

FIG. 4e also illustrates an example synthesis 460 (e.g. modification) of compound C₁ 442 into compounds C₃ 462 and C₄ 464 using the selected subset of action/rule vectors 424i and 424j (e.g. step 408 of process 400) and also the synthesis of compound C₂ 444 into compounds C₅ 466 using the selected subset of action/rule vectors 424m. For example, compound C₃ 462 would be the compound that results from modifying compound C₁ 442 with Rᵢ, such as adding or reforming a bond between atoms of a compound C₁ 442 to form compound C₃ 462. Compound C₄ 464 would be the compound that results from modifying compound C₁ 442 with R^{j}, such as breaking or removing a bond between atoms of a compound C₁ 442 to form compound C₄ 464. Compound C₅ 466 would be the compound that results from modifying compound C₂ 444 with Rₙ₋₁, such as breaking or removing a bond between atoms of a compound C₂ 444 to form compound C₅ 465.

The process 400 may end by outputting a set of candidate compounds and their corresponding subsets of sequences of rules/actions used to modify a compound to form a candidate compound. For example, the set of candidate compounds that are output may include compounds C₃ 462, C₄ 464, and C₅ 465. The sequence of rules used to generate compound C₃ 462 from compound C includes (R₁, Rᵢ), the sequence of rules used to generate C₄ 464 from compound C includes (R₁, Rⱼ), and the sequence of rules used to generate C₅ 465 from compound C includes (R₂, Rₘ), which may also be output from the process 400.

As the process 100 iterates, the encoding ML technique may be updated based on the scoring the output candidate compounds C₃ 462, C₄ 464, and C₅ 465 (e.g. in steps 106-110). The scoring is an indication of how close one or more candidate compounds C₃ 462, C₄ 464, and C₅ 465 are to a compound that exhibits the desired property(ies) and can be used to update the encoding ML technique and hence refine the N-dimensional rule/action space. The encoding ML technique may be rewarded if it generates candidate compounds closer to a compound exhibiting the desired property(ies) or it may be penalised when it generates candidate compounds that exhibit less than the desired property(ies). As the encoding ML technique is updated based on the scoring of process 100, it will refine or adapt the N-dimensional rule/action space and thus the locations of the rule/action vectors and/or proto-rule/action vectors that define candidate compounds will change such that the proto-rule/action vectors a located closer to rule/action vectors that will more likely result in the synthesis of candidate compounds closer to the desired property(ies).

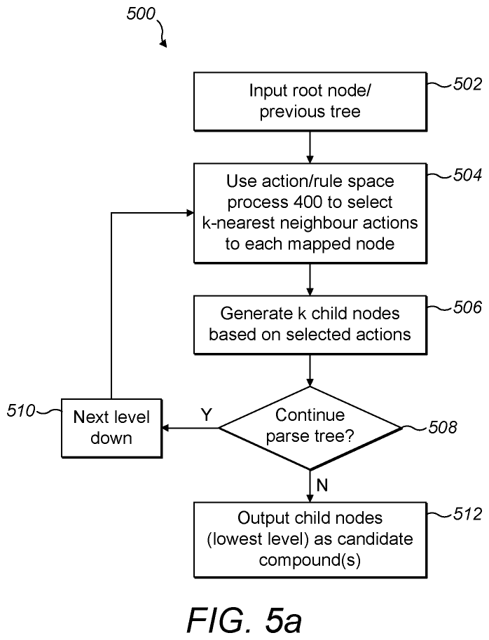

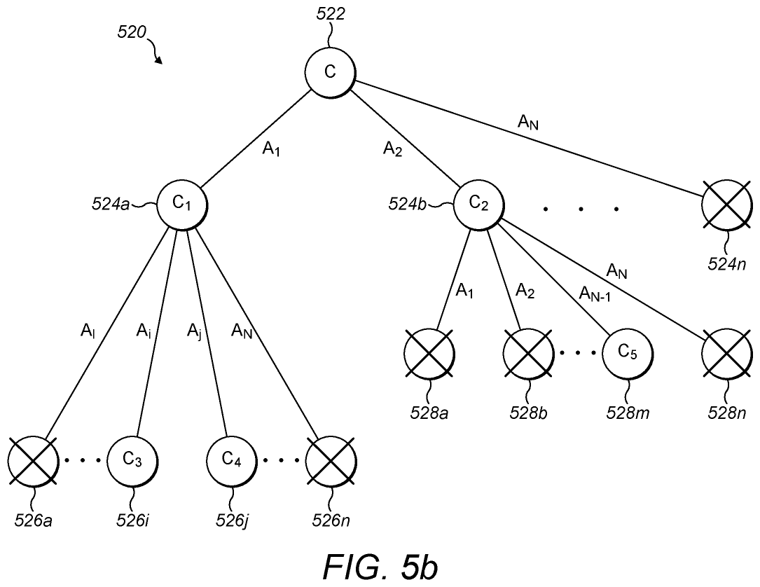

FIG. 5a is a flow diagram illustrating an example process 500 for an tree-encoding ML technique based on FIGS. 3a-4e to generate one or more candidate compounds that may exhibit one or more desired property(ies) according to the invention. The tree-encoding ML technique may be used in step 104 of process 100. The tree-encoding ML technique can efficiently generate a set of candidate compounds for use in step 106 by taking advantage of the tree-based data structure illustrated in FIGS. 3a-3c and the N-dimensional rule/action vector space illustrated in FIGS. 4a-4e. The tree-based structure may be applied to efficiently generate, store and/or maintain the subset sequences of rules that may be used to generate each candidate compound, whilst the N-dimensional rule/action vector space may be applied for efficiently selecting the best subset of rules. The tree-encoding ML technique may be configured (e.g. by a NN structure) to map the set of rules/actions and also to map compounds to an N-dimensional rule/action vector space. As described with reference to FIGS. 4a-4e, the N-dimensional rule/action vector space may be updated or adapted based on scoring the candidate compounds based on the desired property(ies). Thus, in each iteration of steps 104, 106, 110 of process 100, the N-dimensional rule/action vector space of the tree-encoding ML technique may be further refined to more likely enable the selection subsets of rules/actions that may be used to generate candidate compounds that are closer to the desired property(ies).

The process 500 of the tree-encoding ML technique may be based on the following steps: In step 502, a first compound may be input and represented as the root node of a tree-data structure, or a previously generated tree-data structure representing a set of candidate compounds may be input to the tree-encoding ML technique in which each of the leaf nodes represent a candidate compound. In step 504, the encoding-portion of the tree-encoding ML technique may use the N-dimensional rule/action space, in which the set of rules/actions has already been mapped into a set of N-dimensional rules/action vectors, to select a subset of rules/actions for modifying compound in a similar manner as described in process 400 with reference to FIGS. 4a-4e. The first compound represented by the root node may be mapped, by the encoding-portion of the tree-encoding ML technique, into the N-dimensional rule/action space as a compound proto-rule/action vector and the k-nearest neighbour rule/action vectors may be selected, where k>=1. Thus, for the first compound, a subset of rule(s)/action(s) may be generated by demapping the selected k-nearest neighbour rule/action vectors into the corresponding rule(s)/action(s) for modifying said first compound. The selected subset of rule(s)/action(s) may be used, in step 506, to generate one or more candidate compounds, which are represented as child nodes of the root node.

Alternatively or additionally, if a previously generated tree was input, then for each candidate compound or leaf node of the previously generated tree, the candidate compound represented by the leaf node may be mapped, by the encoding-portion of the tree-encoding ML technique, into the N-dimensional rule/action space as a compound proto-rule/action vector and the k-nearest neighbour rule/action vectors may be selected, where k>=1. Thus, for each candidate compound, a selected subset of rule/actions may be generated by demapping the selected k-nearest neighbour rule/action vectors into corresponding rule(s)/action(s) for modifying said each candidate compound to generate further candidate compounds. The selected subset(s) of rule(s)/action(s) may be used, in step 506, to generate one or more candidate compounds, which are represented as child nodes of each leaf node and become new leaf nodes of the tree-based structure.

In step 506, one or more child nodes of the current root node and/or of the leaf nodes may be generated based on the selected one or more subset(s) of rule(s)/action(s). Thus, the tree-encoding ML technique has been able to efficiently select one or more subsets rule(s)/action(s) from the set of rules R₁, . . . , Rₙ for generating one or more child nodes. In step 508, it is determined whether to continue to parse the tree data structure of the tree-encoding ML technique and move down to the next level in the tree and generate further child nodes from the current leaf nodes (current set of child nodes in the lowest level so far generated), or to output the current set of leaf nodes (or a selected set of child nodes) as a selected set of candidate compounds. If it is determined to parse the tree data structure (e.g. ‘Y’), then the process 500 moves to step 510 in which the next level of the tree data structure is generated by repeating steps 504 and 508 but instead using the process 400 on the current set of candidate compounds, which are represented by the newly generated child nodes (or leaf nodes).

In step 508, if it is determined to output a set of candidate compounds (e.g. ‘N’), then, in step 512, the tree-encoding ML technique outputs a set of candidate compound(s) based on the selected set of child nodes or based on the current set of leaf nodes. The tree-encoding ML technique may also output the corresponding sequence(s) of rule(s)/action(s), which can be generated by following a path from the root node of the tree (e.g. the first compound) to each of the selected child/leaf node(s).

As described with reference to FIGS. 4a-4e, the encoding portion of the ML technique may be updated or adapted based on scoring of the candidate compounds against whether they exhibit or are closer to exhibiting the desired property(ies). This adaptation/update will further refine the N-dimensional rule/action vector space and make it more likely that further candidate compounds may be generated that are closer to exhibiting the desired property(ies). In each iteration of steps 104, 106, 110 of process 100, the N-dimensional rule/action vector space of the tree-encoding ML technique may be further refined to more likely enable the selection subsets of rules/actions that may be used to generate candidate compounds that are closer to the desired property(ies).

FIG. 5b is a schematic diagram illustrating an example generation of a set of candidate compounds based on the tree-encoding ML technique as described with reference to FIGS. 3a-5a. The set of candidate compounds may be generated based on a set of rule(s)/action(s) {Rᵢ}ᵢ₌₁ⁿ for modifying compounds, where Rᵢ, is the i-th rule/action for modifying a compound. Each compound or candidate compound may be represented in a tree-data structure 520 by a plurality of nodes 522, 524a-524n, 526a-526n and 528a-528n, in which each non-leaf node may have one or more of a plurality of rule edges R₁, . . . , Rₙ extending therefrom. Each rule edge represents a rule/action from the set of rule(s)/action(s) and connects a parent node to a child node. Each parent node represents a compound and each rule edge from each parent node to a child node represents a rule/action from the set of rule(s)/action(s) rules {Rᵢ}ᵢ₌₁ⁿ that may be performed on the compound represented by the parent node and results in the compound represented by the child node.

In this example, the root node of the tree 520 is created based on a first compound C. Initially, (e.g. see step 502 of process 500), the first compound C may be input to the tree-encoding ML technique, which represents the first compound C as root node 522 of the tree-data structure 520. The tree-encoding ML technique may then select, from the set of rule(s)/action(s) {Rᵢ}ᵢ₌₁ⁿ (e.g. rule edges a subset of rules/actions for modifying compound C in a similar manner as described in process 400 with reference to FIGS. 4a-4e. For example, the tree-encoding ML technique may encode the set of rules/actions {Rᵢ}ᵢ₌₁ⁿ into an N-dimensional rule/action space 422, in which the set of rules/actions {Rᵢ}ᵢ₌₁ⁿ are mapped into a set of N-dimensional rules/action vectors 424a-424n. The first compound C represented by the root node 522 is also encoded into the N-dimensional rule/action space 422, where the first compound C is mapped to a compound proto-rule/action vector 432 in the N-dimensional rule/action space. The tree-encoding ML technique may then select a subset of rules/action vectors 424a-424n that are closest to the compound proto-rule/action vector 432. For example, the k-nearest neighbour rule/action vectors may be selected, where k>=1. In this example, k=2, and the k-nearest neighbour rule/action vectors that are nearest to compound proto-rule/action vector 432 using, by way of example only but is not limited to, distance metrics 434 and 436 are rule/action vectors 424a and 424b, respectively. Thus, for the first compound C, a subset of rule(s)/action(s) may be generated by demapping the selected k-nearest neighbour rule/action vectors 424a and 424b into the corresponding rule(s)/action(s) R₁ and R₂ (or rule edges R₁ and R₂), which can be used to modify said first compound C.

The first compound C can be modified by generating the next level of the tree 520 (e.g. step 506 of process 500) based only on the selected subset of rule(s)/action(s), which in this example includes rule(s)/action(s) R₁ and R₂. The level of the tree 520 can be created by only extending the rule edges corresponding to the selected subset of rule(s) (e.g. rule edges R₁ and R₂) from root node 522 to the corresponding child nodes 524a and 524b. The remaining rule edges {Rᵢ}ᵢ₌₃ⁿ are not extended, so no more child nodes at this level of the tree 520 are created. In this example, the child nodes 524a and 524b represent a set of candidate compounds. Thus, the tree-encoding ML technique has been able to efficiently select one or more subsets rule(s)/action(s) from the set of rules {Rᵢ}ᵢ₌₁ⁿ to generate one or more candidate compound(s) represented by child nodes.

The tree-encoding ML technique may continue to extend the tree 520 to generate further candidate compounds based on the current set of candidate compounds represented by child nodes 524a and 524b (e.g. see step 508 of process 500). In this example, the tree-encoding ML technique extends the tree 520 to the next level to generate further candidate compounds/child nodes. Thus, for each of the child nodes 524a and 524b the tree-encoding ML technique repeats the mapping of compounds and selection of a corresponding subset of rule(s)/action(s) based on k-nearest neighbours.

For example, a first candidate compound represented by child node 524a is encoded into the N-dimensional rule/action space 422, where the first candidate compound 524a is mapped to a first candidate compound proto-rule/action vector 452 in the N-dimensional rule/action space 422. The tree-encoding ML technique may then select a subset of rules/action vectors from the set of rule/action vector(s) 424a-424n that are closest to the first candidate compound proto-rule/action vector 452. For example, the k-nearest neighbour rule/action vectors may be selected, where k>=1. In this example, k=2, and the k-nearest neighbour rule/action vectors that are nearest to the first candidate compound proto-rule/action vector 452 using, by way of example only but is not limited to, distance metrics 456a and 456b are rule/action vectors 424i and 424j, respectively. Thus, for the first candidate compound 524a, the selected subset of rule(s)/action(s) may be generated by demapping the selected k-nearest neighbour rule/action vectors 424i and 424j into the corresponding rule(s)/action(s) Rᵢ and Rⱼ (or rule edges Rᵢ and Rⱼ), which can be used to modify said first candidate compound 524a.

The first candidate compound 524a can be modified by generating the next level of the tree 520 (e.g. step 506 of process 500) based only on the selected subset of rule(s)/action(s), which in this example includes rule(s)/action(s) Rᵢ and Rⱼ. The next level of the tree 520 can be created by only extending the rule edges corresponding to the selected subset of rule(s) (e.g. rule edges Rᵢ and Rⱼ) from child node 524a to the corresponding new child nodes 526i and 526j. The remaining rule edges are not extended, so no more child nodes at this level for this section of the tree 520 are created. In this example, the child nodes 526i and 526j represent another set of candidate compounds C₃ and C₄, respectively.

Similarly, the second candidate compound represented by child node 524b is encoded into the N-dimensional rule/action space 422, where the second candidate compound 524b is mapped to a second candidate compound proto-rule/action vector 454 in the N-dimensional rule/action space 422. The tree-encoding ML technique may then select a subset of rules/action vectors from the set of rule/action vector(s) 424a-424n that are closest to the first candidate compound proto-rule/action vector 452. For example, the k-nearest neighbour rule/action vectors may be selected, where k>=1. In this example, k=2, and the k-nearest neighbour rule/action vectors that are nearest to the first candidate compound proto-rule/action vector 454 using, by way of example only but is not limited to, distance metrics 458 is rule/action vector 424m, respectively. Thus, for the second candidate compound 524b, a subset of rule(s)/action(s) may be generated by demapping the selected k-nearest neighbour rule/action vectors 424m into the corresponding rule(s)/action(s) Rₙ₋₁ (or rule edges Rₙ₋₁), which can be used to modify said second candidate compound 524b.

The second candidate compound 524b can be modified by generating the next level of the tree 520 (e.g. step 506 of process 500) based only on the corresponding selected subset of rule(s)/action(s), which in this example includes rule(s)/action(s) Rₙ₋₁. The next level of the tree 520 can be created by only extending the rule edges corresponding to the selected subset of rule(s) (e.g. rule edges Rₙ₋₁) from child node 524b to corresponding new child node(s) 528m. The remaining rule edges are not extended, so no more child nodes at this level for this section of the tree 520 is created. In this example, the child node(s) 528m represents another set of candidate compounds C₅.

Again, the tree-encoding ML technique may continue to extend the tree 520 to generate further candidate compounds based on the current set(s) of candidate compounds represented at the current level by child nodes 526i, 526j and 528m (e.g. see step 508 of process 500). The tree-encoding ML technique may further extend the tree 520 up to a predetermined number of m levels, where m>=1, (e.g. m iterations of steps 504-508 based on the newly generated nodes of level m−1, where level 0 corresponds to the root node) by repeating the above process steps 504, 506, and 508 for each of the newly generated nodes of the previous level.

Should the tree-encoding ML technique decide (e.g. in step 508 of process 500) to output a set of candidate compounds for assessment and scoring by, for example, step 106 of process 100, then the nodes of the m-th or current level of tree 520 may be used as the set of candidate compounds. The decision may be based on a predetermined number of iterations of generating new levels of the tree 520 being performed or a particular number of candidate compounds have been generated at a particular level of the tree 520. In any event, a set of candidate compounds may be output based on the current set of leaf nodes of the tree 520. For example, nodes 526i, 526j and 528m are the most newly generated nodes of tree 520, thus these nodes 526i, 526j and 528m may be used to output the set of candidate compounds corresponding to C₃, C₄ and C₅. The tree-encoding ML technique may also output the corresponding sequence(s) of rule(s)/action(s), which can be generated by following a path from the root node of the tree (e.g. the first compound) to each of the selected child/leaf node(s). For example, each candidate compound has a corresponding sequence of rule(s)/action(s) that can be applied to first compound C to generate said each candidate compound. These can be generated by parsing or following the path through the tree 520 along the rule edges connecting the root node representing the first compound C with the corresponding child node representing the candidate compound.

For example, the set of candidate compounds that may be output based on tree 520 may include compounds corresponding to the current leaf nodes 526i, 526j and 528m, namely, compounds C₃, C₄, and C₅. The sequence of rules used to generate compound C₃ from compound C includes (R₁, Rᵢ), which are the rule edges that connect node 522 (e.g. compound C) with node 526i (e.g. compound C₃). The sequence of rules used to generate C₄ from compound C includes (R₁, Rⱼ), which are the rule edges that connect node 522 (e.g. compound C) with node 526j (e.g. compound C₄). The sequence of rules used to generate C₅ from compound C includes (R₂, Rₘ), which are the rule edges that connect node 522 (e.g. compound C) with node 528m (e.g. compound C₅).

As the process 100 iterates, the tree-encoding ML technique may be updated based on the scoring the output candidate compounds C₃, C₄, and C₅ (e.g. in steps 106-110). The scoring is an indication of how close one or more candidate compounds C₃, C₄, and C₅ are to a compound that exhibits the desired property(ies) and can be used to update the tree-encoding ML technique and hence refine the corresponding N-dimensional rule/action space 422. The tree-encoding ML technique may be rewarded if it generates candidate compounds closer to a compound exhibiting the desired property(ies) or it may be penalised when it generates candidate compounds that exhibit less than the desired property(ies). As the tree-encoding ML technique is updated based on the scoring of process 100, it will refine or adapt the N-dimensional rule/action space 422 and thus the locations of the rule/action vectors and/or compound proto-rule/action vectors that define candidate compounds will change such that the compound proto-rule/action vectors may be located closer to rule/action vectors that are more likely to result in the synthesis of one or more candidate compounds that are closer to, or exhibit, the desired property(ies).

Once the tree-encoding ML technique has been updated and/or process 100 decides further iterations of steps 104-108 may be necessary, the tree-encoding ML technique may regenerate the tree 520 based on the initial starting compound C and the updated N-dimensional rule/action space 422. This is because the k-nearest neighbour search may select a different set of rules given that the corresponding rule/action vectors and/or compound proto-rule/action vectors will have changed in relation to each other. Alternatively or additionally, if the previous set of candidate compound(s) are much closer to a compound that exhibits the desired property(ies), the tree-encoding ML technique may retain the previous tree-based structure 520 and simply further extend the tree 520. For example, a further m levels of the tree 520 may be generated, or a further predetermined number of new child nodes may be generated. In any event, given the update to the tree-encoding ML technique, the tree-encoding ML technique will make different decisions/selections of the rule(s)/action(s) edges and thus generate different sets of child nodes going forward to yield further improved sets candidate compounds. Both processes 100 and 500 may be further iterated with current or rebuilt tree structures, starting compounds, or even starting with one or more promising candidate compounds and the like.

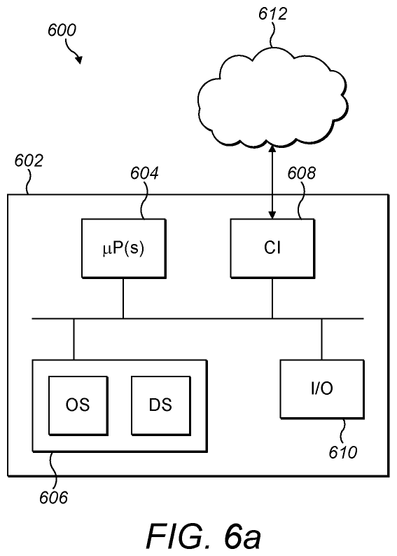

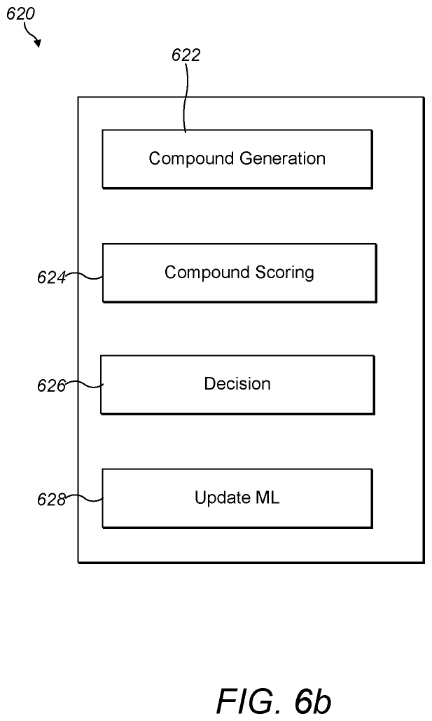

FIG. 6a is a schematic diagram of a computing system 600 comprising a computing apparatus or device 602 according to the invention. The computing apparatus or device 602 may include a processor unit 604, a memory unit 606 and a communication interface 608. The processor unit 604 is connected to the memory unit 606 and the communication interface 608. The processor unit 604 and memory 606 may be configured to implement one or more steps of one or more of the process(es) 100, 300, 400, and/or 500 as described herein. The processor unit 604 may include one or more processor(s), controller(s) or any suitable type of hardware(s) for implementing computer executable instructions to control apparatus 602 according to the invention. The computing apparatus 602 may be connected to a network 612 for communicating and/or operating with other computing apparatus/system(s) (not shown) for implementing the invention accordingly.

FIG. 6b is a schematic diagram illustrating of a example system 620 that may be used to implement one or more aspects of the design and generation of compounds according to the invention and/or implementing one or more of the method(s), apparatus and/or system(s) as described with reference to FIGS. 1a-6a. The system 620 for designing a compound exhibiting one or more desired properties includes a compound generation module or apparatus 622, a compound scoring module or apparatus 624, a decision module or apparatus 626, and an update ML module or apparatus 628, which may be connected together. Although these modules/apparatus are described separately, this is by way of example only, it is to be appreciated by the skilled person that these modules may be combined or even further divided up into further modules/apparatus as the application demands.

In operation, the compound generation module 622 is configured for generating a second compound using the ML technique to modify a first compound based on the desired property(ies) and a set of rules for modifying compounds. The compound scoring module 624 is configured for scoring the second compound based on the desired property(ies). The decision module 626 is configured for determining whether to repeat the generating step based on the scoring. The update ML module 628 is configured for updating the ML technique based on the scoring prior to repeating the generating step.

The system 620 may be further configured to implement the method(s), process(es), apparatus and/or systems as described herein or as described with reference to any of FIGS. 1a to 6a. The system 620 may be configured such that the compound generation module 622, the compound scoring module 624, the decision module 626, and the update ML module 628 are further configured to implement the method(s)/process(es) apparatus and system(s) as described herein or as described with reference to any of FIGS. 1a-6a. For example, the compound generation module or apparatus 622 may be further configured to implement the functionality, method(s), process(es) and/or apparatus associated with generating candidate compounds using ML techniques such as RL techniques, tree-based RL techniques, action-space based techniques, combinations thereof and the like. modifications thereof and/or as described herein or as described with reference to FIGS. 1a-6a. The compound scoring module or device 624 may be further configured to implement the functionality, method(s), process(es) and/or apparatus associated with scoring candidate compounds and the like and/or as described herein or as described with reference to FIGS. 1a-6a. The decision module or device 626 may be further configured to implement the functionality, method(s), process(es) and/or apparatus associated with assessing the candidate compounds based on the scoring and deciding which candidate compounds to proceed with, and also for deciding whether to continue with generating further candidate compounds, or whether the desired properties have been meet for one or more candidate compounds and the like and/or as described herein or as described with reference to FIGS. 1a-6a. The update ML module or device 628 may be further configured to implement the functionality, method(s), process(es) and/or apparatus associated with updating the ML techniques used to generate candidate compound(s) based on the scoring and/or decision(s) associated with the candidate compounds of the current iteration and the like and/or as described herein or as described with reference to FIGS. 1a-6a.

The computing system 600 or system 620 may be a server system, which may comprise a single server or network of servers configured to implement the invention as described herein. In some examples the functionality of the server may be provided by a network of servers distributed across a geographical area, such as a worldwide distributed network of servers, and a user may be connected to an appropriate one of the network of servers based upon a user location.

The systems 600 or 620 may include one or more further modifications, features, steps and/or features of the process(es) 100, 200, 300, 320, 340, 350, 400, 420, 430, 440, 450, 460, 500, 520 and/or apparatus/systems 120, 210, 600, 620, computer-implemented method(s) thereof, and/or modifications thereof, as described with reference to any one or more FIGS. 1a to 6b, and/or as herein described. For example, the compound generation module/device 622, compound scoring module/device 624, decision module/device 626, and/or ML update module/device 628 may be configured to implement one or more further modifications, features, steps and/or features of the process(es) 100, 200, 300, 320, 340, 350, 400, 420, 430, 440, 450, 460, 500, 520 and/or apparatus/systems 120, 210, 600, 620, computer-implemented method(s) thereof, and/or modifications thereof, as described with reference to any one or more FIGS. 1a to 6b, and/or as herein described.

Furthermore, one or more further modifications, features, steps and/or features of the process(es) 100, 200, 300, 320, 340, 350, 400, 420, 430, 440, 450, 460, 500, 520 and/or apparatus/systems 120, 210, 600, 620, computer-implemented method(s) thereof, and/or modifications thereof, as described with reference to any one or more FIGS. 1a to 6b, and/or as herein described may be implemented in hardware and/or software. For example, the method(s) and/or process(es) for designing a compound exhibiting one or more desired properties described with reference to one or more of FIGS. 1a-6b may be implemented in hardware and/or software such as, by way of example only but not limited to, as a computer-implemented method by one or more processor(s)/processor unit(s) or as the application demands. Such apparatus, system(s), process(es) and/or method(s) may be used to generate a ML model including data representative of updating an ML technique as described with respect to the computer-implemented method(s), process(es) 100, 130, 500 and/or apparatus/systems 120, 300, 400, 600, and/or any method(s)/process(es), step(s) of these process(es), as described with reference to any one or more FIGS. 1a to 6, modifications thereof, and/or as described herein and the like. Thus, a ML model may be obtained from apparatus, systems and/or computer-implemented process(es), method(s) as described herein.

The above description discusses embodiments of the invention with reference to a single user for clarity. It will be understood that in practice the system may be shared by a plurality of users, and possibly by a very large number of users simultaneously.

The embodiments described above are fully automatic. In some examples a user or operator of the system may manually instruct some steps of the method to be carried out.

In the described embodiments of the invention the system may be implemented as any form of a computing and/or electronic device or apparatus. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any apparatus or device with processing capability such that it can execute instructions. Those skilled in the art will realise that such processing capabilities are incorporated into many different devices and therefore the term ‘computer’ includes any processing hardware/software, PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method for designing a compound exhibiting one or more desired property(ies) using a machine learning (ML) technique, the method comprising:
generating a second compound using the ML technique to modify a first compound based on the desired property(ies) and a set of rules for modifying compounds;
scoring the second compound based on the desired property(ies);
determining whether to repeat the generating step based on the scoring; and
updating the ML technique based on the scoring prior to repeating the generating step.

2. A computer-implemented method as claimed in claim 1, wherein determining whether to repeat the generating step is based on the scoring indicating the second compound is closer to a compound that exhibits the desired property(ies).

3. A computer-implemented method as claimed in claim 1, wherein determining whether to repeat the generating step is based on the scoring indicating the second compound exhibits the desired property(ies).

4. A computer-implemented method as claimed in claim 1, wherein determining whether to repeat the generating step further comprises determining whether a predetermined number of iterations of repeating the generating step has been achieved.

5. A computer-implemented method as claimed in claim 1, wherein determining whether to repeat the generating step further comprises determining, based on the second compound exhibiting at least one or more of the desired property(ies), whether any further improvements to the second compound are possible.

6. A computer-implemented method as claimed in claim 1, wherein: generating a second compound further comprises generating a set of second compounds; and
scoring the set of second compounds based on the desired property(ies).

7. A computer-implemented method as claimed in claim 6, further comprising:
ranking the set of second compounds based on the scoring, wherein generating a second compound further comprises generating further second compounds based on the topmost ranked set of second compounds.

8. The computer-implemented method according to claim 1, wherein the set of rules further comprises data representative of one or more action(s) associated with modifying compounds

9. The computer-implemented method according to claim 8, wherein the one or more action(s) comprises one or more action(s) from the group of:
an action corresponding to adding a compound fragment or one or more atoms to the compound;
an action corresponding to removing a compound fragment or one or more atoms of the compound;
an action corresponding to breaking or removing a bond between atoms of a compound;
an action corresponding to adding or reforming a bond between atoms of a compound;
any other action associated with modifying a compound to form another compound; and
any other action associated with modifying a compound to form a different compound.

10. The computer-implemented method according to claim 8, wherein the set of rule(s) and/or the one or more action(s) are selected to conform to required structural, physical and/or chemical constraints that ensure any modification(s) to the compound and/or subsequent modified compounds are feasible.

11. The computer-implemented method according to claim 8, wherein the set of rule(s) and/or the one or more action(s) are based on a set of relevantly chemical groups comprising one or more of:
one or more of atom(s);
one or more molecule(s);
one or more other compound(s);
one or more compound fragment(s);
one or more bond(s);
one or more functional group(s); and
one or more chemically relevant aspects of the compound and the like.

12. A computer-implemented method as claimed in claim 8, wherein generating a second compound further comprises generating a tree data structure comprising a plurality of nodes and a plurality of edges, wherein each edge connects a parent node to a child node, wherein a parent node represents a compound and each edge from a parent node to a child node represents an action of the plurality of actions performed on the compound of the parent node that results in the compound of the child node, wherein the root node of the tree is the first compound and subsequent nodes correspond to a set of second compound(s).

13. A computer-implemented method as claimed in claim 12, further comprising expanding the tree data structure based on scoring one or more nodes corresponding to the set of second compound(s).

14. A computer-implemented method as claimed in claim 12, further comprising performing a tree search on the tree data structure to generate a set of second compounds based on a set of one or more actions from the plurality of actions.

15. A computer-implemented method as claimed in claim 12, wherein generating one or more second compound(s) further comprises:
mapping, by the ML technique, the first compound and a set of actions to an N-dimensional action space;
selecting, by the ML technique, a subset of actions in the N-dimensional action space that are nearest neighbour to the first compound when mapped in the N-dimensional action space; and
applying the subset of actions in the N-dimensional space to the first compound to generate a set of one or more second compound(s).

16. A computer-implemented method as claimed in claim 15, wherein generating the set of second compound(s) further comprises selecting nodes associated with the selected set of actions for inclusion into the tree data structure.

17. The computer-implemented method according to claim 1, wherein the desired property(ies) includes one or more from the group of:
the compound docking with another compound to form a stable complex;
the particular property is associated with a ligand docking with a target protein, wherein the compound is the ligand;
the compound docking or binding with one or more target proteins;
the compound having a particular solubility or range of solubilities; and
any other property associated with a compound that can be simulated using computer simulation(s) based on physical movements of atoms and molecules.

18. The computer-implemented method according to claim 1, wherein the score comprises a certainty score, wherein one or more of the second compound(s) has a upper certainty score when those compounds substantially exhibit all of the one or more desired property(ies), one or more of the second compound(s) have a lower certainty score when those compound(s) substantially do not exhibit some of the one or more desired property(ies), and one or more of the second compound(s) have an uncertainty score between the upper certainty score and lower certainty score when those compounds substantially exhibit some of the one or more desired property(ies).

19. The computer-implemented method according to claim 18, wherein the certainty score is a percentage certainty score, wherein the upper certainty score is 100%, the lower certainty score is 0%, and the uncertainty score is between the upper and lower certainty scores.

20. The computer-implemented method according to claim 1, wherein generating the one or more second compound(s) further comprises using a reinforcement learning, RL, technique for selecting the one or more of a plurality of rules for modifying the first compound into a second compound.

21. The computer-implemented method according to claim 1, wherein at least part of the scoring is performed using one or more ML technique(s).

22. The computer-implemented method according to claim 1, wherein the ML technique comprises at least one ML technique or combination of ML technique(s) from the group of:
a recurrent neural network configured for predicting, starting from a first compound, a second compound exhibiting a set of desired property(ies);
convolutional neural network configured for predicting, starting from a first compound, a second compound exhibiting a set of desired property(ies);
reinforcement learning algorithm configured for predicting, starting from a first compound, a second compound exhibiting a set of desired property(ies); and
any neural network structure configured for predicting, starting from a first compound, a second compound exhibiting a set of desired property(ies).

23. The computer-implemented method according to claim 1, wherein scoring a second compound based on the desired property(ies) further comprises:
analysing the second compound against each of the desired property(ies); and
calculating an aggregated score for the second compound based on the analysis.

24. The computer-implemented method according to claim 23, wherein analysing the second compound further comprises performing a computer simulation associated with one or more of the desired property(ies) for the second compound.

25. The computer-implemented method according to claim 23, wherein analysing the second compound further comprises using a knowledge based expert to determine whether the second compound exhibits one or more of the desired property(ies).

26. The computer-implemented method according to claim 1, wherein one or more first compound(s) are input to the ML technique when generating a second compound using the ML technique.

27. The computer-implemented method according to claim 1, wherein generating a second compound using the ML technique further comprises generating a set of second compounds using the ML technique based on the desired property(ies) and the set of rules.

28. An apparatus comprising a processor, a memory unit and a communication interface and computer executable instructions stored in the memory, wherein the processor is connected to the memory unit and the communication interface, wherein the processor and memory are configured to implement the computer-implemented method according to claim 1 by executing the computer executable instructions.

30. A tangible computer-readable medium comprising computer executable instructions, which when executed on a processor, causes the processor to implement the computer-implemented method of claim 1.

31. A machine learning (ML) model comprising data representative of updating an ML technique according to the computer-implemented method of claim 1.

32. A machine learning, ML, model obtained from the computer-implemented method of claim 1.

33. A tangible computer-readable medium comprising computer executable instructions for designing a compound exhibiting one or more desired property(ies) using a machine learning (ML) technique, which when executed on one or more processor(s), causes at least one of the one or more processor(s) to perform at least one of the steps of the method of:
generating a second compound using the ML technique to modify a first compound based on the desired property(ies) and a set of rules for modifying compounds;
scoring the second compound based on the desired property(ies);
determining whether to generate step based on the scoring; and
updating the ML technique based on the scoring prior to repeating the generating step.

34. The computer-readable medium according to claim 33, wherein the computer executable instructions cause the processor to implement one or more steps of the computer-implemented method of claim 2.

35. A system for designing a compound exhibiting one or more desired property(ies) using a machine learning (ML) technique, the system comprising:
a compound generation module configured for generating a second compound using the ML technique to modify a first compound based on the desired property(ies) and a set of rules for modifying compounds;
a compound scoring module configured for scoring the second compound based on the desired property(ies);
a decision module configured for determining whether to repeat the generating step based on the scoring; and
an update ML module configured for updating the ML technique based on the scoring prior to repeating the generating step.

36. The system according to claim 35, wherein the compound generation module, the compound scoring module, the decision module, and the update ML module are further configured and programmed to implement the computer-implemented method of claim 2.
